(19) United States
(12) Patent Application Publication
(10) Pub. No.: US 2011/0244459 A1
(43) Pub. Date: Oct. 6, 2011

(54) METHODS FOR IDENTIFYING ERBB2 ALTERATION IN TUMORS

(75) Inventors: Francois Bertucci, Carnoux-en-Provence (FR); Rebecca Tagett, Marseille (FR); Sabrina Carpentier, Marseille (FR)

(73) Assignees: IPSOGEN, Marseille (FR); INSERM-INSTITUT NATIONAL DE LA SANTE ET DE LA rECHERCHE MEDICALE, Paris Cedex 13 (FR); INSTITUT PAOLI-CALMETTES, Marseille (FR)

(21) Appl. No.: 13/139,072

(22) PCT Filed: Dec. 9, 2009
(86) PCT No.: PCT/IB09/55625

§ 371 Date: Jun. 10, 2011

Related U.S. Application Data
(60) Provisional application No. 61/121,218, filed on Dec. 10, 2008.
(60) Provisional application No. 61/140,110, filed on Dec. 23, 2008.

(51) Int. Cl. C12Q 1/68; G01N 33/53; C40B 40/08

(52) U.S. Cl. 435/6.11; 435/6.14; 435/6.12; 435/7.1; 506/17

(57) ABSTRACT

Methods for identifying ERBB2 (also named HER2) alteration in tumors, in particular cancer, based on the analysis of the expression of at least three genes of the ERBB2 amplicon located within less than one megabase on either side of ERBB2, and eventually of the gene corresponding to the Affymetrix probeset 234046_at (SEQ ID NO: 31), as well as a poynucleotide library useful for the molecular characterization of a cancer including polynucleotide sequences for detecting the genes, and a kit including the library.

### FIELD OF THE INVENTION

The present invention relates to methods for identifying ERBB2 alteration in tumors, in particular cancer, based on the analysis of the over or under expression of polynucleotide sequences in a tissue sample.

### BACKGROUND

The amplification of the ERBB2-region of chromosome 17 results in the constitutive overexpression of the ERBB2 (also named <<HER2>>) oncogene protein and fuels uncontroled tumor growth in approximately 15 to 30% of breast tumors. ERBB2 is considered today as a predictive marker for clinical benefit from trastuzumab, or Herceptin®, a monoclonal antibody directed against the ERBB2 protein, in both primary and metastatic tumors. However current testing methods are inaccurate for as much as 20% of cases and this may lead to missing the benefit of Herceptin® therapy for some patients or, on the contrary, to prescribing unnecessary therapy for others.

Currently, tumors are tested for ERBB2 with 2 main complementary technologies: immunohistochemistry (IHC) which identifies ERBB2 protein expressed in the tumor cells and in situ hybridization (ISH), which quantifies ERBB2 DNA copy number in the cell chromosomes. Some RT-PCR assays, that quantify the amount of ERBB2 mRNA, have also been developed more recently.

There is need of cancer signature showing higher performance, in terms of robustness, specificity and sensibility, for identifying ERBB2 alteration in tumors, in particular cancer.

The Applicant has now defined a new signature predicting ERBB2 status.

### SUMMARY OF THE INVENTION

The authors of the present invention have now discovered, entirely unexpectedly, a signature predicting ERBB2 status, which correlates with the expression of the HER2 protein at cell membrane level. The test, developed on a set of 152 tumors, was validated in 3 independent datasets totaling 152 tumors. The test correlates with the IHC method in 96% of the cases and it resolves 95% of equivocal IHC cases.

Surprisingly, the Inventors found some genes, strongly correlated with ERBB2 IHC.

These genes allow obtaining a signature predicting ERBB2 status in one step with a global performance (sensitivity, specificity, robustness, etc. . . . ) improved compared to the prior 2-steps methods such as those requiring performing the FISH score after performing IHC method.

Furthermore, these genes are independent with the oestrogen receptor (ER) status of the patient. So, there is no need to perform the ER test before performing the test with the genes of the invention.

Finally, the Inventors found the these genes are located in the ERBB2 amplicon, and capture information about DNA amplification.

The method of the invention also reconciles information at the protein, RNA and DNA level. In other words, the information obtained by using the method of the invention reflects the situation at the genomic, transcriptomic, as well as proteomic level.

So, the invention relates to a method for identifying ERBB2 alteration in tumors, in particular cancer, based on the analysis of the over or under expression of genes in a tissue sample, said analysis comprising:
the detection of the expression of a group of genes comprising at least three, or at least four, or at least five, or at least six, or at least seven, or of eight genes of the ERBB2 amplicon, these genes being located within less than one megabase on either side of ERBB2, or
the detection of the expression of a group of genes comprising at least three, or at least four, or at least five, or at least six, or at least seven, or of eight genes of the ERBB2 amplicon, these genes being located within less than one megabase on either side of ERBB2, and the gene corresponding to SEQ ID NO. 31, or
the detection of the expression of a group of genes consisting of at least three, or at least four, or at least five, or at least six, or at least seven, or of eight genes of the ERBB2 amplicon, these genes being located within less than one megabase on either side of ERBB2, or
the detection of the expression of a group of genes consisting of at least three, or at least four, or at least five, or at least six, or at least seven, or of eight genes of the ERBB2 amplicon, these genes being located within less than one megabase on either side of ERBB2, and of gene corresponding to SEQ ID NO. 31.

In a particular aspect of the invention, the method of detection of the expression of the group of genes may comprise, or may consist of at least three, or at least four, or at least five, or at least six, or at least seven, or of eight genes selected among the following genes: ERBB2, C17orf37, GRB7, PERLD1, STARD3, CRKRS, FGFR2, ZRANB1.

In another particular aspect of the invention, the method of detection of the expression of the group of genes may comprise, or may consist of, at least three, or at least four, or at least five, or at least six, or at least seven, or of eight genes selected among the following genes: ERBB2, C17orf37, GRB7, PERLD1, STARD3, CRKRS, FGFR2, ZRANB1, and of the gene corresponding to SEQ ID NO. 31.

In a particular embodiment of the invention, the group of genes may comprise, or may consist of: ERBB2, C17orf37 and GRB7.

In another particular embodiment of the invention, the group of genes may comprise, or may consist of: ERBB2, C17orf37, GRB7, and the gene corresponding to SEQ ID NO. 31.

In another particular aspect of the invention, the group of genes may comprise, or may consist of: ERBB2, C17orf37, GRB7 and PERLD1.

In another particular aspect of the invention, the group of genes may comprise, or may consist of: ERBB2, C17orf37, GRB7 and PERLD1, and the gene corresponding to SEQ ID NO. 31.

In another particular aspect of the invention, the group of genes may comprise, or may consist of: ERBB2, C17orf37, GRB7, PERLD1 and STARD3.

In another particular aspect of the invention, the group of genes may comprise, or may consist of: ERBB2, C17orf37, GRB7, PERLD1 and STARD3 and of the gene corresponding to SEQ ID NO. 31.

In another aspect of the invention, the group of genes may comprise, or may consist of: ERBB2, C17orf37, GRB7, PERLD1, STARD3 and CRKRS.

In another aspect of the invention, the group of genes may comprise, or may consist of: ERBB2, C17orf37, GRB7, PERLD1, STARD3 and CRKRS and of the gene corresponding to SEQ ID NO. 31.

The sequences allowing to detect the genes above mentioned may be of any kind of nucleic acid, as the man skilled in the art surely knows how to detect a gene among other in a tissue sample.

In a particular embodiment of the invention, this detection may be realized by hybridization of polynucleotide sequences from a tissue sample with cDNA total sequence or with cDNA subsequences of said genes, or with primers, or with the following polynucleotide sequences: SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO.29, SEQ ID NO. 30, SEQ ID NO. 31, SEQ ID NO. 32.

In another particular embodiment of the invention, this detection may be realized by hybridization of polynucleotide sequences from a tissue sample with a group of polynucleotide sequences comprising, of consisting of, at least one, or at least two, or at least three, or at least four, or at least five, or at least six, or at least seven, of the following sequences: SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 30, SEQ ID NO. 31, SEQ ID NO. 32.

The polynucleotide sequences SEQ ID NO. 17 to SEQ ID NO. 32 are polynucleotide sequences (also called “probesets”) capable to react with nucleic acid samples of the genes showed in table 1:

TABLE 1

| | SEQ ID NO. | | |
|---|---|---|---|
| Probesets (Affymetrix) | of the | | SEQ ID NO. of |
| HG-U133 plus 2.0 | probeset | gene | the gene |
| 216836_s_at | SEQ ID NO. | ERBB2 | SEQ ID NO. 1 |
| | 17 | | and SEQ ID |
| | | | NO. 2 |
| 55616_at | SEQ ID NO. | PERLD1 | SEQ ID NO. 3 |
| | 18 | | |
| 224447_s_at | SEQ ID NO. | C17orf37 | SEQ ID NO. 4 |
| | 19 | | |
| 210761_s_at | SEQ ID NO. | GRB7 | SEQ ID NO. 5 |
| | 20 | | and SEQ ID |
| | | | NO. 6 |
| 221811_at | SEQ ID NO. | PERLD1 | SEQ ID NO. 3 |
| | 21 | | |
| 202991_at | SEQ ID NO. | STARD3 | SEQ ID NO. 7 |
| | 22 | | |
| 234254_x_at | SEQ ID NO. | ERBB2 | SEQ ID NO. 1 |
| | 23 | | and SEQ ID |
| | | | NO. 2 |
| 210930_s_at | SEQ ID NO. | ERBB2 | SEQ ID NO. 1 |
| | 24 | | and SEQ ID |
| | | | NO. 2 |
| 225691_at | SEQ ID NO. | CRKRS | SEQ ID NO. 8 |
| | 25 | | and 9 |
| 219226_at | SEQ ID NO. | CRKRS | SEQ ID NO. 8 |
| | 26 | | and 9 |
| 240913_at | SEQ ID NO. | FGFR2 | SEQ ID NO. 10 |
| | 27 | | et SEQ ID NO. |
| | | | 11 |
| 225690_at | SEQ ID NO. | CRKRS | SEQ ID NO. 8 |
| | 28 | | and 9 |
| 225130_at | SEQ ID NO. | ZRANB1 | SEQ ID NO. 12 |
| | 29 | | |
| 225694_at | SEQ ID NO. | CRKRS | SEQ ID NO. 8 |
| | 30 | | and 9 |
| 234046_at | SEQ ID NO. | N/A (or the | SEQ ID of the |
| | 31 | name of the | corresponding |
| | | gene that may | gene |
| | | be detected by | |
| | | this probeset | |
| | | sequence) | |
| 213557_at | SEQ ID NO. | CRKRS | SEQ ID NO. 8 |
| | 32 | | and 9 |

The sequences mentioned above are the following ones:

SEQ ID NO. 1:
GTTCCCGGATTTTTGTGGGCGCCTGCCCCGCCCCTCGTCCCCCTGCT
GTGTCCATATATCGAGGCGATAGGGTTAAGGGAAGGCGGACGCCTGA
TGGGTTAATGAGCAAACTGAAGTGTTTTCCATGATCTTTTTTGAGTCG
CAATTGAAGTACCACCTCCCGAGGGTGATTGCTTCCCCATGCGGGGT
AGAACCTTTGCTGTCCTGTTCACCACTCTACCTCCAGCACAGAATTTG
GCTTATGCCTACTCAATGTGAAGATGATGAGGATGAAAACCTTTGTGA
TGATCCACTTCCACTTAATGAATGGTGGCAAAGCAAAGCTATATTCAA
GACCACATGCAAAGCTACTCCCTGAGCAAAGAGTCACAGATAAAACG
GGGGCACCAGTAGAATGGCCAGGACAAACGCAGTGCAGCACAGAGA
CTCAGACCCTGGCAGCCATGCCTGCGCAGGCAGTGATGAGAGTGAC
ATGTACTGTTGTGGACATGCACAAAAGTGAGTGTGCACCGGCACAGA
CATGAAGCTGCGGCTCCCTGCCAGTCCCGAGACCCACCTGGACATG
CTCCGCCACCTCTACCAGGGCTGCCAGGTGGTGCAGGGAAACCTGG
AACTCACCTACCTGCCCACCAATGCCAGCCTGTCCTTCCTGCAGGAT
ATCCAGGAGGTGCAGGGCTACGTGCTCATCGCTCACAACCAAGTGAG
GCAGGTCCCACTGCAGAGGCTGCGGATTGTGCGAGGCACCCAGCTC
TTTGAGGACAACTATGCCCTGGCCGTGCTAGACAATGGAGACCCGCT
GAACAATACCACCCCTGTCACAGGGGCCTCCCCAGGAGGCCTGCGG
GAGCTGCAGCTTCGAAGCCTCACAGAGATCTTGAAAGGAGGGGTCTT
GATCCAGCGGAACCCCCAGCTCTGCTACCAGGACACGATTTTGTGGA
AGGACATCTTCCACAAGAACAACCAGCTGGCTCTCACACTGATAGACA
CCAACCGCTCTCGGGCCTGCCACCCCTGTTCTCCGATGTGTAAGGGC
TCCCGCTGCTGGGGAGAGAGTTCTGAGGATTGTCAGAGCCTGACGC
GCACTGTCTGTGCCGGTGGCTGTGCCCGCTGCAAGGGGCCACTGCC
CACTGACTGCTGCCATGAGCAGTGTGCTGCCGGCTGCACGGGCCCC
AAGCACTCTGACTGCCTGGCCTGCCTCCACTTCAACCACAGTGGCAT
CTGTGAGCTGCACTGCCCAGCCCTGGTCACCTACAACACAGACACGT
TTGAGTCCATGCCCAATCCCGAGGGCCGGTATACATTCGGCGCCAGC
TGTGTGACTGCCTGTCCCTACAACTACCTTTCTACGGACGTGGGATCC
TGCACCCTCGTCTGCCCCCTGCACAACCAAGAGGTGACAGCAGAGGA
TGGAACACAGCGGTGTGAGAAGTGCAGCAAGCCCTGTGCCCGAGTG
TGCTATGGTCTGGGCATGGAGCACTTGCGAGAGGTGAGGGCAGTTAC
CAGTGCCAATATCCAGGAGTTTGCTGGCTGCAAGAAGATCTTTGGGA
GCCTGGCATTTCTGCCGGAGAGCTTTGATGGGGACCCAGCCTCCAAC
ACTGCCCCGCTCCAGCCAGAGCAGCTCCAAGTGTTTGAGACTCTGGA
AGAGATCACAGGTTACCTATACATCTCAGCATGGCCGGACAGCCTGC
CTGACCTCAGCGTCTTCCAGAACCTGCAAGTAATCCGGGGACGAATT
CTGCACAATGGCGCCTACTCGCTGACCCTGCAAGGGCTGGGCATCA
GCTGGCTGGGGCTGCGCTCACTGAGGGAACTGGGCAGTGGACTGGC
CCTCATCCACCATAACACCCACCTCTGCTTCGTGCACACGGTGCCCT
GGGACCAGCTCTTTCGGAACCCGCACCAAGCTCTGCTCCACACTGCC
AACCGGCCAGAGGACGAGTGTGTGGGCGAGGGCCTGGCCTGCCACC
AGCTGTGCGCCCGAGGGCACTGCTGGGGTCCAGGGCCCACCCAGTG
TGTCAACTGCAGCCAGTTCCTTCGGGGCCAGGAGTGCGTGGAGGAAT
GCCGAGTACTGCAGGGGCTCCCCAGGGAGTATGTGAATGCCAGGCA
CTGTTTGCCGTGCCACCCTGAGTGTCAGCCCCAGAATGGCTCAGTGA
CCTGTTTTGGACCGGAGGCTGACCAGTGTGTGGCCTGTGCCCACTAT
AAGGACCCTCCCTTCTGCGTGGCCCGCTGCCCCAGCGGTGTGAAAC
CTGACCTCTCCTACATGCCCATCTGGAAGTTTCCAGATGAGGAGGGC
GCATGCCAGCCTTGCCCCATCAACTGCACCCACTCCTGTGTGGACCT
GGATGACAAGGGCTGCCCCGCCGAGCAGAGAGCCAGCCCTCTGACG
TCCATCATCTCTGCGGTGGTTGGCATTCTGCTGGTCGTGGTCTTGGG
GGTGGTCTTTGGGATCCTCATCAAGCGACGGCAGCAGAAGATCCGGA
AGTACACGATGCGGAGACTGCTGCAGGAAACGGAGCTGGTGGAGCC
GCTGACACCTAGCGGAGCGATGCCCAACCAGGCGCAGATGCGGATC
CTGAAAGAGACGGAGCTGAGGAAGGTGAAGGTGCTTGGATCTGGCG
CTTTTGGCACAGTCTACAAGGGCATCTGGATCCCTGATGGGGAGAAT
GTGAAAATTCCAGTGGCCATCAAAGTGTTGAGGGAAAACACATCCCC
CAAAGCCAACAAAGAAATCTTAGACGAAGCATACGTGATGGCTGGTG
TGGGCTCCCCATATGTCTCCCGCCTTCTGGGCATCTGCCTGACATCC
ACGGTGCAGCTGGTGACACAGCTTATGCCCTATGGCTGCCTCTTAGA
CCATGTCCGGGAAAACCGCGGACGCCTGGGCTCCCAGGACCTGCTG
AACTGGTGTATGCAGATTGCCAAGGGGATGAGCTACCTGGAGGATGT
GCGGCTCGTACACAGGGACTTGGCCGCTCGGAACGTGCTGGTCAAG
AGTCCCAACCATGTCAAAATTACAGACTTCGGGCTGGCTCGGCTGCT
GGACATTGACGAGACAGAGTACCATGCAGATGGGGGCAAGGTGCCC
ATCAAGTGGATGGCGCTGGAGTCCATTCTCCGCCGGCGGTTCACCCA
CCAGAGTGATGTGTGGAGTTATGGTGTGACTGTGTGGGAGCTGATGA
CTTTTGGGGCCAAACCTTACGATGGGATCCCAGCCCGGGAGATCCCT
GACCTGCTGGAAAAGGGGGAGCGGCTGCCCCAGCCCCCCATCTGCA
CCATTGATGTCTACATGATCATGGTCAAATGTTGGATGATTGACTCTG
AATGTCGGCCAAGATTCCGGGAGTTGGTGTCTGAATTCTCCCGCATG
GCCAGGGACCCCCAGCGCTTTGTGGTCATCCAGAATGAGGACTTGG
GCCCAGCCAGTCCCTTGGACAGCACCTTCTACCGCTCACTGCTGGAG
GACGATGACATGGGGGACCTGGTGGATGCTGAGGAGTATCTGGTAC
CCCAGCAGGGCTTCTTCTGTCCAGACCCTGCCCCGGGCGCTGGGGG
CATGGTCCACCACAGGCACCGCAGCTCATCTACCAGGAGTGGCGGT
GGGGACCTGACACTAGGGCTGGAGCCCTCTGAAGAGGAGGCCCCCA
GGTCTCCACTGGCACCCTCCGAAGGGGCTGGCTCCGATGTATTTGAT
GGTGACCTGGGAATGGGGGCAGCCAAGGGGCTGCAAAGCCTCCCCA
CACATGACCCCAGCCCTCTACAGCGGTACAGTGAGGACCCCACAGTA
CCCCTGCCCTCTGAGACTGATGGCTACGTTGCCCCCCTGACCTGCAG
CCCCCAGCCTGAATATGTGAACCAGCCAGATGTTCGGCCCCAGCCCC
CTTCGCCCCGAGAGGGCCCTCTGCCTGCTGCCCGACCTGCTGGTGC
CACTCTGGAAAGGCCCAAGACTCTCTCCCCAGGGAAGAATGGGGTCG
TCAAAGACGTTTTTGCCTTTGGGGGTGCCGTGGAGAACCCCGAGTAC
TTGACACCCCAGGGAGGAGCTGCCCCTCAGCCCCACCCTCCTCCTG
CCTTCAGCCCAGCCTTCGACAACCTCTATTACTGGGACCAGGACCCA
CCAGAGCGGGGGGCTCCACCCAGCACCTTCAAAGGGACACCTACGG
CAGAGAACCCAGAGTACCTGGGTCTGGACGTGCCAGTGTGAACCAGA
AGGCCAAGTCCGCAGAAGCCCTGATGTGTCCTCAGGGAGCAGGGAA
GGCCTGACTTCTGCTGGCATCAAGAGGTGGGAGGGCCCTCCGACCA
CTTCCAGGGGAACCTGCCATGCCAGGAACCTGTCCTAAGGAACCTTC
CTTCCTGCTTGAGTTCCCAGATGGCTGGAAGGGGTCCAGCCTCGTTG
GAAGAGGAACAGCACTGGGGAGTCTTTGTGGATTCTGAGGCCCTGCC
CAATGAGACTCTAGGGTCCAGTGGATGCCACAGCCCAGCTTGGCCCT
TTCCTTCCAGATCCTGGGTACTGAAAGCCTTAGGGAAGCTGGCCTGA
GAGGGGAAGCGGCCCTAAGGGAGTGTCTAAGAACAAAAGCGACCCA
TTCAGAGACTGTCCCTGAAACCTAGTACTGCCCCCCATGAGGA
AGGAACAGCAATGGTGTCAGTATCCAGGCTTTGTACAGAGTGCTTTTC
TGTTTAGTTTTTACTTTTTTTGTTTTGTTTTTTTAAAGATGAAATAAAGA
CCCAGGGGGAGAATGGGTGTTGTATGGGGAGGCAAGTGTGGGGGGTC
CTTCTCCACACCCACTTTGTCCATTTGCAAATATATTTTGGAAAACAGC
TA
SEQ ID NO. 2:
GGAGGAGGTGGAGGAGGAGGGCTGCTTGAGGAAGTATAAGAATGAA
GTTGTGAAGCTGAGATTCCCCTCCATTGGGACCGGAGAAACCAGGGG
AGCCCCCCGGGCAGCCGCGCGCCCCTTCCCACGGGGCCCTTTACTG
CGCCGCGCGCCCGGCCCCCACCCCTCGCAGCACCCCGCGCCCCGC
GCCCTCCCAGCCGGGTCCAGCCGGAGCCATGGGGCCGGAGCCGCA
GTGAGCACCATGGAGCTGGCGGCCTTGTGCCGCTGGGGGCTCCTCC
TCGCCCTCTTGCCCCCCGGAGCCGCGAGCACCCAAGTGTGCACCGG
CACAGACATGAAGCTGCGGCTCCCTGCCAGTCCCGAGACCCACCTG
GACATGCTCCGCCACCTCTACCAGGGCTGCCAGGTGGTGCAGGGAA
ACCTGGAACTCACCTACCTGCCCACCAATGCCAGCCTGTCCTTCCTG
CAGGATATCCAGGAGGTGCAGGGCTACGTGCTCATCGCTCACAACCA
AGTGAGGCAGGTCCCACTGCAGAGGCTGCGGATTGTGCGAGGCACC
CAGCTCTTTGAGGACAACTATGCCCTGGCCGTGCTAGACAATGGAGA
CCCGCTGAACAATACCACCCCTGTCACAGGGGCCTCCCCAGGAGGC
CTGCGGGAGCTGCAGCTTCGAAGCCTCACAGAGATCTTGAAAGGAGG
GGTCTTGATCCAGCGGAACCCCCAGCTCTGCTACCAGGACACGATTT
TGTGGAAGGACATCTTCCACAAGAACAACCAGCTGGCTCTCACACTG
ATAGACACCAACCGCTCTCGGGCCTGCCACCCCTGTTCTCCGATGTG
TAAGGGCTCCCGCTGCTGGGGAGAGAGTTCTGAGGATTGTCAGAGC
CTGACGCGCACTGTCTGTGCCGGTGGCTGTGCCCGCTGCAAGGGGC
CACTGCCCACTGACTGCTGCCATGAGCAGTGTGCTGCCGGCTGCAC
GGGCCCCAAGCACTCTGACTGCCTGGCCTGCCTCCACTTCAACCACA
GTGGCATCTGTGAGCTGCACTGCCCAGCCCTGGTCACCTACAACACA
GACACGTTTGAGTCCATGCCCAATCCCGAGGGCCGGTATACATTCGG
CGCCAGCTGTGTGACTGCCTGTCCCTACAACTACCTTTCTACGGACG
TGGGATCCTGCACCCTCGTCTGCCCCCTGCACAACCAAGAGGTGACA
GCAGAGGATGGAACACAGCGGTGTGAGAAGTGCAGCAAGCCCTGTG
CCCGAGTGTGCTATGGTCTGGGCATGGAGCACTTGCGAGAGGTGAG
GGCAGTTACCAGTGCCAATATCCAGGAGTTTGCTGGCTGCAAGAAGA
TCTTTGGGAGCCTGGCATTTCTGCCGGAGAGCTTTGATGGGGACCCA
GCCTCCAACACTGCCCCGCTCCAGCCAGAGCAGCTCCAAGTGTTTGA
GACTCTGGAAGAGATCACAGGTTACCTATACATCTCAGCATGGCCGG
ACAGCCTGCCTGACCTCAGCGTCTTCCAGAACCTGCAAGTAATCCGG
GGACGAATTCTGCACAATGGCGCCTACTCGCTGACCCTGCAAGGGCT
GGGCATCAGCTGGCTGGGGCTGCGCTCACTGAGGGAACTGGGCAGT
GGACTGGCCCTCATCCACCATAACACCCACCTCTGCTTCGTGCACAC
GGTGCCCTGGGACCAGCTCTTTCGGAACCCGCACCAAGCTCTGCTCC
ACACTGCCAACCGGCCAGAGGACGAGTGTGTGGGCGAGGGCCTGGC
CTGCCACCAGCTGTGCGCCCGAGGGCACTGCTGGGGTCCAGGGCCC
ACCCAGTGTGTCAACTGCAGCCAGTTCCTTCGGGGCCAGGAGTGCGT
GGAGGAATGCCGAGTACTGCAGGGGCTCCCCAGGGAGTATGTGAAT
GCCAGGCACTGTTTGCCGTGCCACCCTGAGTGTCAGCCCCAGAATGG
CTCAGTGACCTGTTTTGGACCGGAGGCTGACCAGTGTGTGGCCTGTG
CCCACTATAAGGACCCTCCCTTCTGCGTGGCCCGCTGCCCCAGCGGT
GTGAAACCTGACCTCTCCTACATGCCCATCTGGAAGTTTCCAGATGAG
GAGGGCGCATGCCAGCCTTGCCCCATCAACTGCACCCACTCCTGTGT
GGACCTGGATGACAAGGGCTGCCCCGCCGAGCAGAGAGCCAGCCCT
CTGACGTCCATCATCTCTGCGGTGGTTGGCATTCTGCTGGTCGTGGT
CTTGGGGGTGGTCTTTGGGATCCTCATCAAGCGACGGCAGCAGAAGA
TCCGGAAGTACACGATGCGGAGACTGCTGCAGGAAACGGAGCTGGT
GGAGCCGCTGACACCTAGCGGAGCGATGCCCAACCAGGCGCAGATG
CGGATCCTGAAAGAGACGGAGCTGAGGAAGGTGAAGGTGCTTGGAT
CTGGCGCTTTTGGCACAGTCTACAAGGGCATCTGGATCCCTGATGGG
GAGAATGTGAAAATTCCAGTGGCCATCAAAGTGTTGAGGGAAAACAC
ATCCCCCAAAGCCAACAAAGAAATCTTAGACGAAGCATACGTGATGG
CTGGTGTGGGCTCCCCATATGTCTCCCGCCTTCTGGGCATCTGCCTG
ACATCCACGGTGCAGCTGGTGACACAGCTTATGCCCTATGGCTGCCT
CTTAGACCATGTCCGGGAAAACCGCGGACGCCTGGGCTCCCAGGAC
CTGCTGAACTGGTGTATGCAGATTGCCAAGGGGATGAGCTACCTGGA
GGATGTGCGGCTCGTACACAGGGACTTGGCCGCTCGGAACGTGCTG
GTCAAGAGTCCCAACCATGTCAAAATTACAGACTTCGGGCTGGCTCG
GCTGCTGGACATTGACGAGACAGAGTACCATGCAGATGGGGGCAAG
GTGCCCATCAAGTGGATGGCGCTGGAGTCCATTCTCCGCCGGCGGTT
CACCCACCAGAGTGATGTGTGGAGTTATGGTGTGACTGTGTGGGAGC
TGATGACTTTTGGGGCCAAACCTTACGATGGGATCCCAGCCCGGGAG
ATCCCTGACCTGCTGGAAAAGGGGGAGCGGCTGCCCCAGCCCCCCA
TCTGCACCATTGATGTCTACATGATCATGGTCAAATGTTGGATGATTG
ACTCTGAATGTCGGCCAAGATTCCGGGAGTTGGTGTCTGAATTCTCC
CGCATGGCCAGGGACCCCCAGCGCTTTGTGGTCATCCAGAATGAGG
ACTTGGGCCCAGCCAGTCCCTTGGACAGCACCTTCTACCGCTCACTG
CTGGAGGACGATGACATGGGGGACCTGGTGGATGCTGAGGAGTATC
TGGTACCCCAGCAGGGCTTCTTCTGTCCAGACCCTGCCCCGGGCGCT
GGGGGCATGGTCCACCACAGGCACCGCAGCTCATCTACCAGGAGTG
GCGGTGGGGACCTGACACTAGGGCTGGAGCCCTCTGAAGAGGAGGC
CCCCAGGTCTCCACTGGCACCCTCCGAAGGGGCTGGCTCCGATGTAT
TTGATGGTGACCTGGGAATGGGGGCAGCCAAGGGGCTGCAAAGCCT
CCCCACACATGACCCCAGCCCTCTACAGCGGTACAGTGAGGACCCCA
CAGTACCCCTGCCCTCTGAGACTGATGGCTACGTTGCCCCCCTGACC
TGCAGCCCCCAGCCTGAATATGTGAACCAGCCAGATGTTCGGCCCCA
GCCCCCTTCGCCCCGAGAGGGCCCTCTGCCTGCTGCCCGACCTGCT
GGTGCCACTCTGGAAAGGCCCAAGACTCTCTCCCCAGGGAAGAATGG
GGTCGTCAAAGACGTTTTTGCCTTTGGGGGTGCCGTGGAGAACCCCG
AGTACTTGACACCCCAGGGAGGAGCTGCCCCTCAGCCCCACCCTCCT
CCTGCCTTCAGCCCAGCCTTCGACAACCTCTATTACTGGGACCAGGA
CCCACCAGAGCGGGGGGCTCCACCCAGCACCTTCAAAGGGACACCT
ACGGCAGAGAACCCAGAGTACCTGGGTCTGGACGTGCCAGTGTGAA
CCAGAAGGCCAAGTCCGCAGAAGCCCTGATGTGTCCTCAGGGAGCA
GGGAAGGCCTGACTTCTGCTGGCATCAAGAGGTGGGAGGGCCCTCC
GACCACTTCCAGGGGAACCTGCCATGCCAGGAACCTGTCCTAAGGAA
CCTTCCTTCCTGCTTGAGTTCCCAGATGGCTGGAAGGGGTCCAGCCT
CGTTGGAAGAGGAACAGCACTGGGGAGTCTTTGTGGATTCTGAGGCC
CTGCCCAATGAGACTCTAGGGTCCAGTGGATGCCACAGCCCAGCTTG
GCCCTTTCCTTCCAGATCCTGGGTACTGAAAGCCTTAGGGAAGCTGG
CCTGAGAGGGGAAGCGGCCCTAAGGGAGTGTCTAAGAACAAAAGCG
ACCCATTCAGAGACTGTCCCTGAAACCTAGTACTGCCCCCCATGAGG
AAGGAACAGCAATGGTGTCAGTATCCAGGCTTTGTACAGAGTGCTTTT
CTGTTTAGTTTTTACTTTTTTTGTTTTGTTTTTTTAAAGATGAAATAAAG
ACCCAGGGGGAGAATGGGTGTTGTATGGGGAGGCAAGTGTGGGGGGT
CCTTCTCCACACCCACTTTGTCCATTTGCAAATATATTTTGGAAAACA
GCTA
SEQ ID NO. 3:
ATACTCCTAAGCTCCTCCCCCGGCGGCGAGCCAGGGAGAAAGGATG
GCCGGCCTGGCGGCGCGGTTGGTCCTGCTAGCTGGGGCAGCGGCG
CTGGCGAGCGGCTCCCAGGGCGACCGTGAGCCGGTGTACCGCGACT
GCGTACTGCAGTGCGAAGAGCAGAACTGCTCTGGGGGCGCTCTGAA
TCACTTCCGCTCCCGCCAGCCAATCTACATGAGTCTAGCAGGCTGGA
CCTGTCGGGACGACTGTAAGTATGAGTGTATGTGGGTCACCGTTGGG
CTCTACCTCCAGGAAGGTCACAAAGTGCCTCAGTTCCATGGCAAGTG
GCCCTTCTCCCGGTTCCTGTTCTTTCAAGAGCCGGCATCGGCCGTGG
CCTCGTTTCTCAATGGCCTGGCCAGCCTGGTGATGCTCTGCCGCTAC
CGCACCTTCGTGCCAGCCTCCTCCCCCATGTACCACACCTGTGTGGC
CTTCGCCTGGGTGTCCCTCAATGCATGGTTCTGGTCCACAGTTTTCCA
CACCAGGGACACTGACCTCACAGAGAAAATGGACTACTTCTGTGCCT
CCACTGTCATCCTACACTCAATCTACCTGTGCTGCGTCAGGACCGTG
GGGCTGCAGCACCCAGCTGTGGTCAGTGCCTTCCGGGCTCTCCTGC
TGCTCATGCTGACCGTGCACGTCTCCTACCTGAGCCTCATCCGCTTC
GACTATGGCTACAACCTGGTGGCCAACGTGGCTATTGGCCTGGTCAA
CGTGGTGTGGTGGCTGGCCTGGTGCCTGTGGAACCAGCGGCGGCTG
CCTCACGTGCGCAAGTGCGTGGTGGTGGTCTTGCTGCTGCAGGGGC
TGTCCCTGCTCGAGCTGCTTGACTTCCCACCGCTCTTCTGGGTCCTG
GATGCCCATGCCATCTGGCACATCAGCACCATCCCTGTCCACGTCCT
CTTTTTCAGCTTTCTGGAAGATGACAGCCTGTACCTGCTGAAGGAATC
AGAGGACAAGTTCAAGCTGGACTGAAGACCTTGGAGCGAGTCTGCCC
CAGTGGGGATCCTGCCCCCGCCCTGCTGGCCTCCCTTCTCCCCTCAA
CCCTTGAGATGATTTTCTCTTTTCAACTTCTTGAACTTGGACATGAAGG
ATGTGGGCCCAGAATCATGTGGCCAGCCCACCCCCTGTTGGCCCTCA
CCAGCCTTGGAGTCTGTTCTAGGGAAGGCCTCCCAGCATCTGGGACT
CGAGAGTGGGCAGCCCCTCTACCTCCTGGAGCTGAACTGGGGTGGA
ACTGAGTGTGCTCTTAGCTCTACCGGGAGGACAGCTGCCTGTTTCCT
CCCCATCAGCCTCCTCCCCACATCCCCAGCTGCCTGGCTGGGTCCTG
AAGCCCTCTGTCTACCTGGGAGACCAGGGACCACAGGCCTTAGGGAT
ACAGGGGGTCCCCTTCTGTTACCACCCCCCACCCTCCTCCAGGACAC
CACTAGGTGGTGCTGGATGCTTGTTCTTTGGCCAGCCAAGGTTCACG
GCGATTCTCCCCATGGGATCTTGAGGGACCAAGCTGCTGGGATTGGG
AAGGAGTTTCACCCTGACCATTGCCCTAGCCAGGTTCCCAGGAGGCC
TCACCATACTCCCTTTCAGGGCCAGGGCTCCAGCAAGCCCAGGGCAA
GGATCCTGTGCTGCTGTCTGGTTGAGAGCCTGCCACCGTGTGTCGGG
AGTGTGGGCCAGGCTGAGTGCATAGGTGACAGGGCCGTGAGCATGG
GCCTGGGTGTGTGTGAGCTCAGGCCTAGGTGCGCAGTGTGGAGACG
GGTGTTGTCGGGGAAGAGGTGTGGCTTCAAAGTGTGTGTGTGCAGG
GGGTGGGTGTGTTAGCGTGGGTTAGGGGAACGTGTGTGCGCGTGCT
GGTGGGCATGTGAGATGAGTGACTGCCGGTGAATGTGTCCACAGTTG
AGAGGTTGGAGCAGGATGAGGGAATCCTGTCACCATCAATAATCACT
TGTGGAGCGCCAGCTCTGCCCAAGGCGCCACCTGGGCGGACAGCCA
GGAGCTCTCCATGGCCAGGCTGCCTGTGTGCATGTTCCCTGTCTGGT
GCCCCTTTGCCCGCCTCCTGCAAACCTCACAGGGTCCCCACACAACA
GTGCCCTCCAGAAGCAGCCCCTCGGAGGCAGAGGAAGGAAAATGGG
GATGGCTGGGGCTCTCTCCATCCTCCTTTTCTCCTTGCCTTCGCATGG
CTGGCCTTCCCCTCCAAAACCTCCATTCCCCTGCTGCCAGCCCCTTT
GCCATAGCCTGATTTTGGGGAGGAGGAAGGGGCGATTTGAGGGAGA
AGGGGAGAAAGCTTATGGCTGGGTCTGGTTTCTTCCCTTCCCAGAGG
GTCTTACTGTTCCAGGGTGGCCCCAGGGCAGGCAGGGGCCACACTA
TGCCTGCGCCCTGGTAAAGGTGACCCCTGCCATTTACCAGCAGCCCT
GGCATGTTCCTGCCCCACAGGAATAGAATGGAGGGAGCTCCAGAAAC
TTTCCATCCCAAAGGCAGTCTCCGTGGTTGAAGCAGACTGGATTTTTG
CTCTGCCCCTGACCCCTTGTCCCTCTTTGAGGGAGGGGAGCTATGCT
AGGACTCCAACCTCAGGGACTCGGGTGGCCTGCGCTAGCTTCTTTTG
ATACTGAAAACTTTTAAGGTGGGAGGGTGGCAAGGGATGTGCTTAATA
AATCAATTCCAAGCCTCAAAAAAAAAAAAAAAAAAAAAA
SEQ ID NO. 4:
GTCACACCCGGAAGCAGGGGCCCGAGCGGAGCCGGCCGCGATGAG
CGGGGAGCCGGGGCAGACGTCCGTAGCGCCCCCTCCCGAGGAGGT
CGAGCCGGGCAGTGGGGTCCGCATCGTGGTGGAGTACTGTGAACCC
TGCGGCTTCGAGGCGACCTACCTGGAGCTGGCCAGTGCTGTGAAGG
AGCAGTATCCGGGCATCGAGATCGAGTCGCGCCTCGGGGGCACAGG
TGCCTTTGAGATAGAGATAAATGGACAGCTGGTGTTCTCCAAGCTGGA
GAATGGGGGCTTTCCCTATGAGAAAGATCTCATTGAGGCCATCCGAA
GAGCCAGTAATGGAGAAACCCTAGAAAAGATCACCAACAGCCGTCCT
CCCTGCGTCATCCTGTGACTGCACAGGACTCTGGGTTCCTGCTCTGT
TCTGGGGTCCAAACCTTGGTCTCCCTTTGGTCCTGCTGGGAGCTCCC
CCTGCCTCTTTCCCCTACTTAGCTCCTTAGCAAAGAGACCCTGGCCTC
CACTTTGCCCTTTGGGTACAAAGAAGGAATAGAAGATTCCGTGGCCTT
GGGGGCAGGAGAGAGACACTCTCCATGAACACTTCTCCAGCCACCTC
ATACCCCCTTCCCAGGGTAAGTGCCCACGAAAGCCCAGTCCACTCTT
CGCCTCGGTAATACCTGTCTGATGCCACAGATTTTATTTATTCTCCCCT
AACCCAGGGCAATGTCAGCTATTGGCAGTAAAGTGGCGCTACAAACA
CTAAAAAAAAAAAAAAAAAAAAAAAAAAAAA
SEQ ID NO. 5:
CGTCTCCCTCCCTGAAGACGTGGTCCCAGCCGGGTGTCCTGACGCTC
GGGGTTCAGGACAAGGGCACACAACTGGTTCCGTTAAGCCCCTCTCT
TGCTCAGACGCCATGGAGCTGGATCTGTCTCCACCTCATCTTAGCAG
CTCTCCGGAAGACCTTTGCCCAGCCCCTGGGACCCCTCCTGGGACTC
CCCGGCCCCCTGATACCCCTCTGCCTGAGGAGGTAAAGAGGTCCCA
GCCTCTCCTCATCCCAACCACCGGCAGGAAACTTCGAGAGGAGGAGA
GGCGTGCCACCTCCCTCCCCTCTATCCCCAACCCCTTCCCTGAGCTC
TGCAGTCCTCCCTCACAGAGCCCAATTCTCGGGGGCCCCTCCAGTGC
AAGGGGGCTGCTCCCCCGCGATGCCAGCCGCCCCCATGTAGTAAAG
GTGTACAGTGAGGATGGGGCCTGCAGGTCTGTGGAGGTGGCAGCAG
GTGCCACAGCTCGCCACGTGTGTGAAATGCTGGTGCAGCGAGCTCAC
GCCTTGAGCGACGAGACCTGGGGGCTGGTGGAGTGCCACCCCCACC
TAGCACTGGAGCGGGGTTTGGAGGACCACGAGTCCGTGGTGGAAGT
GCAGGCTGCCTGGCCCGTGGGCGGAGATAGCCGCTTCGTCTTCCGG
AAAAACTTCGCCAAGTACGAACTGTTCAAGAGCTCCCCACACTCCCTG
TTCCCAGAAAAAATGGTCTCCAGCTGTCTCGATGCACACACTGGTATA
TCCCATGAAGACCTCATCCAGAACTTCCTGAATGCTGGCAGCTTTCCT
GAGATCCAGGGCTTTCTGCAGCTGCGGGGTTCAGGACGGAAGCTTTG
GAAACGCTTTTTCTGCTTCTTGCGCCGATCTGGCCTCTATTACTCCAC
CAAGGGCACCTCTAAGGATCCGAGGCACCTGCAGTACGTGGCAGAT
GTGAACGAGTCCAACGTGTACGTGGTGACGCAGGGCCGCAAGCTCT
ACGGGATGCCCACTGACTTCGGTTTCTGTGTCAAGCCCAACAAGCTT
CGAAATGGCCACAAGGGGCTTCGGATCTTCTGCAGTGAAGATGAGCA
GAGCCGCACCTGCTGGCTGGCTGCCTTCCGCCTCTTCAAGTACGGG
GTGCAGCTGTACAAGAATTACCAGCAGGCACAGTCTCGCCATCTGCA
TCCATCTTGTTTGGGCTCCCCACCCTTGAGAAGTGCCTCAGATAATAC
CCTGGTGGCCATGGACTTCTCTGGCCATGCTGGGCGTGTCATTGAGA
ACCCCCGGGAGGCTCTGAGTGTGGCCCTGGAGGAGGCCCAGGCCTG
GAGGAAGAAGACAAACCACCGCCTCAGCCTGCCCATGCCAGCCTCC
GGCACGAGCCTCAGTGCAGCCATCCACCGCACCCAACTCTGGTTCCA
CGGGCGCATTTCCCGTGAGGAGAGCCAGCGGCTTATTGGACAGCAG
GGCTTGGTAGACGGCCTGTTCCTGGTCCGGGAGAGTCAGCGGAACC
CCCAGGGCTTTGTCCTCTCTTTGTGCCACCTGCAGAAAGTGAAGCATT
ATCTCATCCTGCCGAGCGAGGAGGAGGGCCGCCTGTACTTCAGCATG
GATGATGGCCAGACCCGCTTCACTGACCTGCTGCAGCTCGTGGAGTT
CCACCAGCTGAACCGCGGCATCCTGCCGTGCTTGCTGCGCCATTGCT
GCACGCGGGTGGCCCTCTGACCAGGCCGTGGACTGGCTCATGCCTC
AGCCCGCCTTCAGGCTGCCCGCCGCCCCTCCACCCATCCAGTGGAC
TCTGGGGCGCGGCCACAGGGGACGGGATGAGGAGCGGGAGGGTTC
CGCCACTCCAGTTTTCTCCTCTGCTTCTTTGCCTCCCTCAGATAGAAA
ACAGCCCCCACTCCAGTCCACTCCTGACCCCTCTCCTCAAGGGAAGG
CCTTGGGTGGCCCCCTCTCCTTCTCCTAGCTCTGGAGGTGCTGCTCT
AGGGCAGGGAATTATGGGAGAAGTGGGGGCAGCCCAGGCGGTTTCA
CGCCCCACACTTTGTACAGACCGAGAGGCCAGTTGATCTGCTCTGTT
TTATACTAGTGACAATAAAGATTATTTTTTGATACAAAAAAAAAAAAAAA
AAAAAAAAA
SEQ ID NO. 6:
TTTTAGTTTCCTTGGGCCTGGAATCTGGACACACAGGGCTCCCCCCC
GCCTCTGACTTCTCTGTCCGAAGTCGGGACACCCTCCTACCACCTGT
AGAGAAGCGGGAGTGGATCTGAAATAAAATCCAGGAATCTGGGGGTT
CCTAGACGGAGCCAGACTTCGGAACGGGTGTCCTGCTACTCCTGCTG
GGGCTCCTCCAGGACAAGGGCACACAACTGGTTCCGTTAAGCCCCTC
TCTTGCTCAGACGCCATGGAGCTGGATCTGTCTCCACCTCATCTTAGC
AGCTCTCCGGAAGACCTTTGCCCAGCCCCTGGGACCCCTCCTGGGA
CTCCCCGGCCCCCTGATACCCCTCTGCCTGAGGAGGTAAAGAGGTCC
CAGCCTCTCCTCATCCCAACCACCGGCAGGAAACTTCGAGAGGAGGA
GAGGCGTGCCACCTCCCTCCCCTCTATCCCCAACCCCTTCCCTGAGC
TCTGCAGTCCTCCCTCACAGAGCCCAATTCTCGGGGGCCCCTCCAGT
GCAAGGGGGCTGCTCCCCCGCGATGCCAGCCGCCCCCATGTAGTAA
AGGTGTACAGTGAGGATGGGGCCTGCAGGTCTGTGGAGGTGGCAGC
AGGTGCCACAGCTCGCCACGTGTGTGAAATGCTGGTGCAGCGAGCT
CACGCCTTGAGCGACGAGACCTGGGGGCTGGTGGAGTGCCACCCCC
ACCTAGCACTGGAGCGGGGTTTGGAGGACCACGAGTCCGTGGTGGA
AGTGCAGGCTGCCTGGCCCGTGGGCGGAGATAGCCGCTTCGTCTTC
CGGAAAAACTTCGCCAAGTACGAACTGTTCAAGAGCTCCCCACACTC
CCTGTTCCCAGAAAAAATGGTCTCCAGCTGTCTCGATGCACACACTG
GTATATCCCATGAAGACCTCATCCAGAACTTCCTGAATGCTGGCAGCT
TTCCTGAGATCCAGGGCTTTCTGCAGCTGCGGGGTTCAGGACGGAAG
CTTTGGAAACGCTTTTTCTGCTTCTTGCGCCGATCTGGCCTCTATTAC
TCCACCAAGGGCACCTCTAAGGATCCGAGGCACCTGCAGTACGTGGC
AGATGTGAACGAGTCCAACGTGTACGTGGTGACGCAGGGCCGCAAG
CTCTACGGGATGCCCACTGACTTCGGTTTCTGTGTCAAGCCCAACAA
GCTTCGAAATGGCCACAAGGGGCTTCGGATCTTCTGCAGTGAAGATG
AGCAGAGCCGCACCTGCTGGCTGGCTGCCTTCCGCCTCTTCAAGTAC
GGGGTGCAGCTGTACAAGAATTACCAGCAGGCACAGTCTCGCCATCT
GCATCCATCTTGTTTGGGCTCCCCACCCTTGAGAAGTGCCTCAGATAA
TACCCTGGTGGCCATGGACTTCTCTGGCCATGCTGGGCGTGTCATTG
AGAACCCCCGGGAGGCTCTGAGTGTGGCCCTGGAGGAGGCCCAGGC
CTGGAGGAAGAAGACAAACCACCGCCTCAGCCTGCCCATGCCAGCCT
CCGGCACGAGCCTCAGTGCAGCCATCCACCGCACCCAACTCTGGTTC
CACGGGCGCATTTCCCGTGAGGAGAGCCAGCGGCTTATTGGACAGC
AGGGCTTGGTAGACGGCCTGTTCCTGGTCCGGGAGAGTCAGCGGAA
CCCCCAGGGCTTTGTCCTCTCTTTGTGCCACCTGCAGAAAGTGAAGC
ATTATCTCATCCTGCCGAGCGAGGAGGAGGGCCGCCTGTACTTCAGC
ATGGATGATGGCCAGACCCGCTTCACTGACCTGCTGCAGCTCGTGGA
GTTCCACCAGCTGAACCGCGGCATCCTGCCGTGCTTGCTGCGCCATT
GCTGCACGCGGGTGGCCCTCTGACCAGGCCGTGGACTGGCTCATGC
CTCAGCCCGCCTTCAGGCTGCCCGCCGCCCCTCCACCCATCCAGTG
GACTCTGGGGCGCGGCCACAGGGGACGGGATGAGGAGCGGGAGGG
TTCCGCCACTCCAGTTTTCTCCTCTGCTTCTTTGCCTCCCTCAGATAG
AAAACAGCCCCCACTCCAGTCCACTCCTGACCCCTCTCCTCAAGGGA
AGGCCTTGGGTGGCCCCCTCTCCTTCTCCTAGCTCTGGAGGTGCTGC
TCTAGGGCAGGGAATTATGGGAGAAGTGGGGGCAGCCCAGGCGGTT
TCACGCCCCACACTTTGTACAGACCGAGAGGCCAGTTGATCTGCTCT
GTTTTATACTAGTGACAATAAAGATTATTTTTTGATACAAAAAAAAAAAA
AAAAAAAAAAAA
SEQ ID NO. 7:
GGCGCTACTGAGGCCGCGGAGCCGGACTGCGGTTGGGGCGGGAAG
AGCCGGGGCCGTGGCTGACATGGAGCAGCCCTGCTGCTGAGGCCGC
GCCCTCCCCGCCCTGAGGTGGGGGCCCACCAGGATGAGCAAGCTGC
CCAGGGAGCTGACCCGAGACTTGGAGCGCAGCCTGCCTGCCGTGGC
CTCCCTGGGCTCCTCACTGTCCCACAGCCAGAGCCTCTCCTCGCACC
TCCTTCCGCCGCCTGAGAAGCGAAGGGCCATCTCTGATGTCCGCCGC
ACCTTCTGTCTCTTCGTCACCTTCGACCTGCTCTTCATCTCCCTGCTC
TGGATCATCGAACTGAATACCAACACAGGCATCCGTAAGAACTTGGA
GCAGGAGATCATCCAGTACAACTTTAAAACTTCCTTCTTCGACATCTTT
GTCCTGGCCTTCTTCCGCTTCTCTGGACTGCTCCTAGGCTATGCCGT
GCTGCGGCTCCGGCACTGGTGGGTGATTGCGGTCACGACGCTGGTG
TCCAGTGCATTCCTCATTGTCAAGGTCATCCTCTCTGAGCTGCTCAGC
AAAGGGGCATTTGGCTACCTGCTCCCCATCGTCTCTTTTGTCCTCGCC
TGGTTGGAGACCTGGTTCCTTGACTTCAAAGTCCTACCCCAGGAAGC
TGAAGAGGAGCGATGGTATCTTGCCGCCCAGGTTGCTGTTGCCCGTG
GACCCCTGCTGTTCTCCGGTGCTCTGTCCGAGGGACAGTTCTATTCA
CCCCCAGAATCCTTTGCAGCGTCTGACAATGAATCAGATGAAGAAGTT
GCTGGGAAGAAAAGTTTCTCTGCTCAGGAGCGGGAGTACATCCGCCA
GGGGAAGGAGGCCACGGCAGTGGTGGACCAGATCTTGGCCCAGGAA
GAGAACTGGAAGTTTGAGAAGAATAATGAATATGGGGACACCGTGTA
CACCATTGAAGTTCCCTTTCACGGCAAGACGTTTATCCTGAAGACCTT
CCTGCCCTGTCCTGCGGAGCTCGTGTACCAGGAGGTGATCCTGCAG
CCCGAGAGGATGGTGCTGTGGAACAAGACAGTGACTGCCTGCCAGAT
CCTGCAGCGAGTGGAAGACAACACCCTCATCTCCTATGACGTGTCTG
CAGGGGCTGCGGGCGGCGTGGTCTCCCCAAGGGACTTCGTGAATGT
CCGGCGCATTGAGCGGCGCAGGGACCGATACTTGTCATCAGGGATC
GCCACCTCACACAGTGCCAAGCCCCCGACGCACAAATATGTCCGGG
GAGAGAATGGCCCTGGGGGCTTCATCGTGCTCAAGTCGGCCAGTAAC
CCCCGTGTTTGCACCTTTGTCTGGATTCTTAATACAGATCTCAAGGGC
CGCCTGCCCCGGTACCTCATCCACCAGAGCCTCGCGGCCACCATGTT
TGAATTTGCCTTTCACCTGCGACAGCGCATCAGCGAGCTGGGGGCCC
GGGCGTGACTGTGCCCCCTCCCACCCTGCGGGCCAGGGTCCTGTCG
CCACCACTTCCAGAGCCAGAAAGGGTGCCAGTTGGGCTCGCACTGC
CCACATGGGACCTGGCCCCAGGCTGTCACCCTCCACCGAGCCACGC
AGTGCCTGGAGTTGACTGACTGAGCAGGCTGTGGGGTGGAGCACTG
GACTCCGGGGCCCCACTGGCTGGAGGAAGTGGGGTCTGGCCTGTTG
ATGTTTACATGGCGCCCTGCCTCCTGGAGGACCAGATTGCTCTGCCC
CACCTTGCCAGGGCAGGGTCTGGGCTGGGCACCTGACTTGGCTGGG
GAGGACCAGGGCCCTGGGCAGGGCAGGGCAGCCTGTCACCCGTGT
GAAGATGAAGGGGCTCTTCATCTGCCTGCGCTCTCGTCGGTTTTTTTA
GGATTATTGAAAGAGTCTGGGACCCTTGTTGGGGAGTGGGTGGCAGG
TGGGGGTGGGCTGCTGGCCATGAATCTCTGCCTCTCCCAGGCTGTCC
CCCTCCTCCCAGGGCCTCCTGGGGGACCTTTGTATTAAGCCAATTAA
AAACATGAATTTAAAAAAAAAAAAAAAAAAAAAAAAAAAAAAAAAAAAAA
AAAAAAAAAAAA
SEQ ID NO. 8:
AAGTGCCGTTTCGGTTTAATCTAGTGTGTGACTGGGTCTGTGTGAGG
GAGAGAGTGTGTGTGGTGTGGAGGTGAAACGGAGGCAAGAAAGGGG
GCTACCTCAGGAGCGAGGGACAAAGGGGGCGTGAGGCACCTAGGCC
GCGGCACCCCGGCGACAGGAAGCCGTCCTGAACCGGGCTACCGGGT
AGGGGAAGGGCCCGCGTAGTCCTCGCAGGGCCCCAGAGCTGGAGTC
GGCTCCACAGCCCCGGGCCGTCGGCTTCTCACTTCCTGGACCTCCC
CGGCGCCCGGGCCTGAGGACTGGCTCGGCGGAGGGAGAAGAGGAA
ACAGACTTGAGCAGCTCCCCGTTGTCTCGCAACTCCACTGCCGAGGA
ACTCTCATTTCTTCCCTCGCTCCTTCACCCCCCACCTCATGTAGAAGG
GTGCTGAGGCGTCGGGAGGGAGGAGGAGCCTGGGCTACCGTCCCT
GCCCTCCCCACCCCCTTCCCGGGGCGCTTTGGTGGGCGTGGAGTTG
GGGTTGGGGGGGTGGGTGGGGGTTGCTTTTTGGAGTGCTGGGGAAC
TTTTTTCCCTTCTTCAGGTCAGGGGAAAGGGAATGCCCAATTCAGAGA
GACATGGGGGCAAGAAGGACGGGAGTGGAGGAGCTTCTGGAACTTT
GCAGCCGTCATCGGGAGGCGGCAGCTCTAACAGCAGAGAGCGTCAC
CGCTTGGTATCGAAGCACAAGCGGCATAAGTCCAAACACTCCAAAGA
CATGGGGTTGGTGACCCCCGAAGCAGCATCCCTGGGCACAGTTATCA
AACCTTTGGTGGAGTATGATGATATCAGCTCTGATTCCGACACCTTCT
CCGATGACATGGCCTTCAAACTAGACCGAAGGGAGAACGACGAACGT
CGTGGATCAGATCGGAGCGACCGCCTGCACAAACATCGTCACCACCA
GCACAGGCGTTCCCGGGACTTACTAAAAGCTAAACAGACCGAAAAAG
AAAAAAGCCAAGAAGTCTCCAGCAAGTCGGGATCGATGAAGGACCGG
ATATCGGGAAGTTCAAAGCGTTCGAATGAGGAGACTGATGACTATGG
GAAGGCGCAGGTAGCCAAAAGCAGCAGCAAGGAATCCAGGTCATCC
AAGCTCCACAAGGAGAAGACCAGGAAAGAACGGGAGCTGAAGTCTG
GGCACAAAGACCGGAGTAAAAGTCATCGAAAAAGGGAAACACCCAAA
AGTTACAAAACAGTGGACAGCCCAAAACGGAGATCCAGGAGCCCCCA
CAGGAAGTGGTCTGACAGCTCCAAACAAGATGATAGCCCCTCGGGAG
CTTCTTATGGCCAAGATTATGACCTTAGTCCCTCACGATCTCATACCT
CGAGCAATTATGACTCCTACAAGAAAAGTCCTGGAAGTACCTCGAGAA
GGCAGTCGGTCAGTCCCCCTTACAAGGAGCCTTCGGCCTACCAGTCC
AGCACCCGGTCACCGAGCCCCTACAGTAGGCGACAGAGATCTGTCA
GTCCCTATAGCAGGAGACGGTCGTCCAGCTACGAAAGAAGTGGCTCT
TACAGCGGGCGATCGCCCAGTCCCTATGGTCGAAGGCGGTCCAGCA
GCCCTTTCCTGAGCAAGCGGTCTCTGAGTCGGAGTCCACTCCCCAGT
AGGAAATCCATGAAGTCCAGAAGTAGAAGTCCTGCATATTCAAGACAT
TCATCTTCTCATAGTAAAAAGAAGAGATCCAGTTCACGCAGTCGTCAT
TCCAGTATCTCACCTGTCAGGCTTCCACTTAATTCCAGTCTGGGAGCT
GAACTCAGTAGGAAAAAGAAGGAAAGAGCAGCTGCTGCTGCTGCAGC
AAAGATGGATGGAAAGGAGTCCAAGGGTTCACCTGTATTTTTGCCTAG
AAAAGAGAACAGTTCAGTAGAGGCTAAGGATTCAGGTTTGGAGTCTAA
AAAGTTACCCAGAAGTGTAAAATTGGAAAAATCTGCCCCAGATACTGA
ACTGGTGAATGTAACACATCTAAACACAGAGGTAAAAAATTCTTCAGA
TACAGGGAAAGTAAAGTTGGATGAGAACTCCGAGAAGCATCTTGTTAA
AGATTTGAAAGCACAGGGAACAAGAGACTCTAAACCCATAGCACTGAA
AGAGGAGATTGTTACTCCAAAGGAGACAGAAACATCAGAAAAGGAGA
CCCCTCCACCTCTTCCCACAATTGCTTCTCCCCCACCCCCTCTACCAA
CTACTACCCCTCCACCTCAGACACCCCCTTTGCCACCTTTGCCTCCAA
TACCAGCTCTTCCACAGCAACCACCTCTGCCTCCTTCTCAGCCAGCAT
TTAGTCAGGTTCCTGCTTCCAGTACTTCAACTTTGCCCCCTTCTACTCA
CTCAAAGACATCTGCTGTGTCCTCTCAGGCAAATTCTCAGCCCCCTGT
ACAGGTTTCTGTGAAGACTCAAGTATCTGTAACAGCTGCTATTCCACA
CCTGAAAACTTCAACGTTGCCTCCTTTGCCCCTCCCACCCTTATTACC
TGGAGATGATGACATGGATAGTCCAAAAGAAACTCTTCCTTCAAAACC
TGTGAAGAAAGAGAAGGAACAGAGGACACGTCACTTACTCACAGACC
TTCCTCTCCCTCCAGAGCTCCCTGGTGGAGATCTGTCTCCCCCAGAC
TCTCCAGAACCAAAGGCAATCACACCACCTCAGCAACCATATAAAAAG
AGACCAAAAATTTGTTGTCCTCGTTATGGAGAAAGAAGACAAACAGAA
AGCGACTGGGGGAAACGCTGTGTGGACAAGTTTGACATTATTGGGAT
TATTGGAGAAGGAACCTATGGCCAAGTATATAAAGCCAAGGACAAAGA
CACAGGAGAACTAGTGGCTCTGAAGAAGGTGAGACTAGACAATGAGA
AAGAGGGCTTCCCAATCACAGCCATTCGTGAAATCAAAATCCTTCGTC
AGTTAATCCACCGAAGTGTTGTTAACATGAAGGAAATTGTCACAGATA
AACAAGATGCACTGGATTTCAAGAAGGACAAAGGTGCCTTTTACCTTG
TATTTGAGTATATGGACCATGACTTAATGGGACTGCTAGAATCTGGTT
TGGTGCACTTTTCTGAGGACCATATCAAGTCGTTCATGAAACAGCTAA
TGGAAGGATTGGAATACTGTCACAAAAAGAATTTCCTGCATCGGGATA
TTAAGTGTTCTAACATTTTGCTGAATAACAGTGGGCAAATCAAACTAGC
AGATTTTGGACTTGCTCGGCTCTATAACTCTGAAGAGAGTCGCCCTTA
CACAAACAAAGTCATTACTTTGTGGTACCGACCTCCAGAACTACTGCT
AGGAGAGGAACGTTACACACCAGCCATAGATGTTTGGAGCTGTGGAT
GTATTCTTGGGGAACTATTCACAAAGAAGCCTATTTTTCAAGCCAATCT
GGAACTGGCTCAGCTAGAACTGATCAGCCGACTTTGTGGTAGCCCTT
GTCCAGCTGTGTGGCCTGATGTTATCAAACTGCCCTACTTCAACACCA
TGAAACCGAAGAAGCAATATCGAAGGCGTCTACGAGAAGAATTCTCTT
TCATTCCTTCTGCAGCACTTGATTTATTGGACCACATGCTGACACTAG
ATCCTAGTAAGCGGTGCACAGCTGAACAGACCCTACAGAGCGACTTC
CTTAAAGATGTCGAACTCAGCAAAATGGCTCCTCCAGACCTCCCCCAC
TGGCAGGATTGCCATGAGTTGTGGAGTAAGAAACGGCGACGTCAGCG
ACAAAGTGGTGTTGTAGTCGAAGAGCCACCTCCATCCAAAACTTCTCG
AAAAGAAACTACCTCAGGGACAAGTACTGAGCCTGTGAAGAACAGCA
GCCCAGCACCACCTCAGCCTGCTCCTGGCAAGGTGGAGTCTGGGGC
TGGGGATGCAATAGGCCTTGCTGACATCACACAACAGCTGAATCAAA
GTGAATTGGCAGTGTTATTAAACCTGCTGCAGAGCCAAACCGACCTG
AGCATCCCTCAAATGGCACAGCTGCTTAACATCCACTCCAACCCAGA
GATGCAGCAGCAGCTGGAAGCCCTGAACCAATCCATCAGTGCCCTGA
CGGAAGCTACTTCCCAGCAGCAGGACTCAGAGACCATGGCCCCAGA
GGAGTCTTTGAAGGAAGCACCCTCTGCCCCAGTGATCCTGCCTTCAG
CAGAACAGACGACCCTTGAAGCTTCAAGCACACCAGCTGACATGCAG
AATATATTGGCAGTTCTCTTGAGTCAGCTGATGAAAACCCAAGAGCCA
GCAGGCAGTCTGGAGGAAAACAACAGTGACAAGAACAGTGGGCCAC
AGGGGCCCCGAAGAACTCCCACAATGCCACAGGAGGAGGCAGCAGA
GAAGAGGCCCCCTGAGCCCCCCGGACCTCCACCGCCGCCACCTCCA
CCCCCTCTGGTTGAAGGCGATCTTTCCAGCGCCCCCCAGGAGTTGAA
CCCAGCCGTGACAGCCGCCTTGCTGCAACTTTTATCCCAGCCTGAAG
CAGAGCCTCCTGGCCACCTGCCACATGAGCACCAGGCCTTGAGACCA
ATGGAGTACTCCACCCGACCCCGTCCAAACAGGACTTATGGAAACAC
TGATGGGCCTGAAACAGGGTTCAGTGCCATTGACACTGATGAACGAA
ACTCTGGTCCAGCCTTGACAGAATCCTTGGTCCAGACCCTGGTGAAG
AACAGGACCTTCTCAGGCTCTCTGAGCCACCTTGGGGAGTCCAGCAG
TTACCAGGGCACAGGGTCAGTGCAGTTTCCAGGGGACCAGGACCTC
CGTTTTGCCAGGGTCCCCTTAGCGTTACACCCGGTGGTCGGGCAACC
ATTCCTGAAGGCTGAGGGAAGCAGCAATTCTGTGGTACATGCAGAGA
CCAAATTGCAAAACTATGGGGAGCTGGGGCCAGGAACCACTGGGGC
CAGCAGCTCAGGAGCAGGCCTTCACTGGGGGGGCCCAACTCAGTCT
TCTGCTTATGGAAAACTCTATCGGGGGCCTACAAGAGTCCCACCAAG
AGGGGGAAGAGGGAGAGGAGTTCCTTACTAACCCAGAGACTTCAGTG
TCCTGAAAGATTCCTTTCCTATCCATCCTTCCATCCAGTTCTCTGAATC
TTTAATGAAATCATTTGCCAGAGCGAGGTAATCATCTGCATTTGGCTA
CTGCAAAGCTGTCCGTTGTATTCCTTGCTCACTTGCTACTAGCAGGCG
ACTTACGAAATAATGATGTTGGCACCAGTTCCCCCTGGATGGGCTATA
GCCAGAACATTTACTTCAACTCTACCTTAGTAGATACAAGTAGAGAATA
TGGAGAGGATCATTACATTGAAAAGTAAATGTTTTATTAGTTCATTGCC
TGCACTTACTGATCGGAAGAGAGAAAGAACAGTTTCAGTATTGAGATG
GCTCAGGAGAGGCTCTTTGATTTTTAAAGTTTTGGGGTGGGGGATTGT
GTGTGGTTTCTTTCTTTTGAATTTTAATTTAGGTGTTTTGGGTTTTTTTC
CTTTAAAGAGAATAGTGTTCACAAAATTTGAGCTGCTCTTTGGCTTTTG
CTATAAGGGAAACAGAGTGGCCTGGCTGATTTGAATAAATGTTTCTTT
CCTCTCCACCATCTCACATTTTGCTTTTAAGTGAACACTTTTTCCCCAT
TGAGCATCTTGAACATACTTTTTTTCCAAATAAATTACTCATCCTTAAAG
TTTACTCCACTTTGACAAAAGATACGCCCTTCTCCCTGCACATAAAGC
AGGTTGTAGAACGTGGCATTCTTGGGCAAGTAGGTAGACTTTACCCA
GTCTCTTTCCTTTTTTGCTGATGTGTGCTCTCTCTCTCTCTTTCTCTCT
CTCTCTCTCTCTCTCTCTCTCTCTCTCTCTCTCTGTCTCGCTTGCTCGC
TCTCGCTGTTTCTCTCTCTTTGAGGCATTTGTTTGGAAAAAATCGTTGA
GATGCCCAAGAACCTGGGATAATTCTTTACTTTTTTTGAAATAAAGGAA
AGGAAATTCAGACTCTTACATTGTTCTCTGTAACTCTTCAATTCTAAAA
TGTTTTGTTTTTTAAACCATGTTCTGATGGGGAAGTTGATTTGTAAGTG
TGGACAGCTTGGACATTGCTGCTGAGCTGTGGTTAGAGATGATGCCT
CCATTCCTAGAGGGCTAATAACAGCATTTAGCATATTGTTTACACATAT
ATTTTTATGTCAAAAAAAAAACAAAAACCTTTCAAACAGAGCATTGTGA
TATTGTCAAAGAGAAAAACAAATCCTGAAGATACATGGAAATGTAACC
TAGTTTAGGGTGGGTATTTTTCTGAAGATACATCAATACCTGACCTTTT
TTAAAAAAATAATTTTAAAACAGCATACTGTGAGGAAGAACAGTATTGA
CATACCCACATCCCAGCATGTGTACCCTGCCAGTTCTTTTAGGGATTT
TTCCTCCAAAGAGATTTGGATTTGGTTTTGGTAAAAGGGGTTAAATTGT
GCTTCCAGGCAAGAACTTTGCCTTATCATAAACAGGAAATGAAAAAGG
GAAGGGCTGTCAGGATGGGATAATTTGGGAGGCTTCTCATTCTGGCT
TCTATTTCTATGTGAGTACCAGCATATAGAGTGTTTTAAAAACAGATAC
ATGTCATATAATTTATCTGCACAGACTTAGACCTTCAGGAAACATAGGT
TAAGCCCCCTTTTACAAAGAAAAAGTAAACATACTTCAGCATCTTGGA
GGGTAGTTTTCAAAACTCAAGTTTCATGTTTCAATGCCAAGTTCTTATT
TTAAAAAATAAAATCTACTTATAAGAGAAAGGTGCATTACTTAAAAAAA
AAAAACTTTAAAGAAATGAAAGAAGAACCCTCTTCAGATACTTACTTGA
AGACTGTTTTCCCCTGTTAATGAGATATAGCTAGATATCGGTGTGTGT
ATTTCTTTATTATTCTCTGGTTTTTGATCTGGCCTTGCCTCCAGGGCCA
AACACTGATTTAGAAAGAGAGCCTTCTAGCTATTTTGGCATTGATGGC
TTTTTATACCAGTGTGTCCAGTTAGATTTACTAGGCTTACTGACATGCT
ATTGGTAAATCGCATTAAAGTTCATCTGAACCTTCTGTCTGTTGACTTC
TTAGTCCTCAGACATGGGCCTTTGTGTTTTAGAATATTTGAATTTGAGT
TATTGGGCCCCACTCCCTGTTTTTTATTAAAGAACGTGAGCCTGGGAT
ACTTTCAGAAGTATCTGTTCAATGAAAAAAAGTTGGTTTCCCATCAAAT
ATGAATAAAATTCTCTATATATTTCATTGTATTTTGGTTATCAGCAGTCA
TCAATAATGTTTTTCCCTCCCCTCTCCCACCTCTTATTTTTAATTATGCC
AAATATCCTAAATAATATACTTAAGCCTCCATTCCCTCATCCCTACTAG
GGAAGGGGGTGAGTGTATGTGTGAGTGTATGTGTATGTATGATCCCA
TCTCACCCCCACCCCCATTTTGGGAGTCTTTTAAAATGAAAACAAAGT
TTGGTAGTTTTGACTATTTCTAAAAGCAGAGGAGAAAAAAAAACTTATT
TAAATATCCTGGAATCTGTATGGAGGAAGAAAAGGTATTTGTTAATTTT
TCAGTTACGTTATCTATAAACATGATGGAAGTAAAGGTTTGGCAGAATT
TCACCTTGACTATTTGAAAATTACAGACCCAATTAATTCCATTCAAAAG
TGGTTTTCGTTTTGTTTTAATTATTGTACAATGAGAGATATTGTCTATTA
AATACATTATTTTGAACAGATGAGAAATCTGATTCTGTTCATGAGTGGG
AGGCAAAACTGGTTTGACCGTGATCATTTTTGTGGTTTTGAAAACAAAT
ATACTTGACCCAGTTTCCTTAGTTTTTTCTTCAACTGTCCATAGGAACG
ATAAGTATTTGAAAGCAACATCAAATCTATACGTTTAAAGCAGGGCAG
TTAGCACAAATTTGCAAGTAGAACTTCTATTAGCTTATGCCATAGACAT
CACCCAACCACTTGTATGTGTGTGTGTATATATAATATGCATATATAGT
TACCGTGCTAAAATGGTTACCAGCAGGTTTTGAGAGAGAATGCTGCAT
CAGAAAAGTGTCAGTTGCCACCTCATTCTCCCTGATTTAGGTTCCTGA
CACTGATTCCTTTCTCTCTCGTTTTTGACCCCCATTGGGTGTATCTTGT
CTATGTACAGATATTTTGTAATATATTAAATTTTTTTCTTTCAGTTTATA
AAAATGGAAAGTGGAGATTGGAAAATTAAATATTTCCTGTTACTATACCA
AAAAAAAAAAAAAAAAAAAA
SEQ ID NO. 9:
AAGTGCCGTTTCGGTTTAATCTAGTGTGTGACTGGGTCTGTGTGAGG
GAGAGAGTGTGTGTGGTGTGGAGGTGAAACGGAGGCAAGAAAGGGG
GCTACCTCAGGAGCGAGGGACAAAGGGGGCGTGAGGCACCTAGGCC
GCGGCACCCCGGCGACAGGAAGCCGTCCTGAACCGGGCTACCGGGT
AGGGGAAGGGCCCGCGTAGTCCTCGCAGGGCCCCAGAGCTGGAGTC
GGCTCCACAGCCCCGGGCCGTCGGCTTCTCACTTCCTGGACCTCCC
CGGCGCCCGGGCCTGAGGACTGGCTCGGCGGAGGGAGAAGAGGAA
ACAGACTTGAGCAGCTCCCCGTTGTCTCGCAACTCCACTGCCGAGGA
ACTCTCATTTCTTCCCTCGCTCCTTCACCCCCCACCTCATGTAGAAGG
GTGCTGAGGCGTCGGGAGGGAGGAGGAGCCTGGGCTACCGTCCCT
GCCCTCCCCACCCCCTTCCCGGGGCGCTTTGGTGGGCGTGGAGTTG
GGGTTGGGGGGGTGGGTGGGGGTTGCTTTTTGGAGTGCTGGGGAAC
TTTTTTCCCTTCTTCAGGTCAGGGGAAAGGGAATGCCCAATTCAGAGA
GACATGGGGGCAAGAAGGACGGGAGTGGAGGAGCTTCTGGAACTTT
GCAGCCGTCATCGGGAGGCGGCAGCTCTAACAGCAGAGAGCGTCAC
CGCTTGGTATCGAAGCACAAGCGGCATAAGTCCAAACACTCCAAAGA
CATGGGGTTGGTGACCCCCGAAGCAGCATCCCTGGGCACAGTTATCA
AACCTTTGGTGGAGTATGATGATATCAGCTCTGATTCCGACACCTTCT
CCGATGACATGGCCTTCAAACTAGACCGAAGGGAGAACGACGAACGT
CGTGGATCAGATCGGAGCGACCGCCTGCACAAACATCGTCACCACCA
GCACAGGCGTTCCCGGGACTTACTAAAAGCTAAACAGACCGAAAAAG
AAAAAAGCCAAGAAGTCTCCAGCAAGTCGGGATCGATGAAGGACCGG
ATATCGGGAAGTTCAAAGCGTTCGAATGAGGAGACTGATGACTATGG
GAAGGCGCAGGTAGCCAAAAGCAGCAGCAAGGAATCCAGGTCATCC
AAGCTCCACAAGGAGAAGACCAGGAAAGAACGGGAGCTGAAGTCTG
GGCACAAAGACCGGAGTAAAAGTCATCGAAAAAGGGAAACACCCAAA
AGTTACAAAACAGTGGACAGCCCAAAACGGAGATCCAGGAGCCCCCA
CAGGAAGTGGTCTGACAGCTCCAAACAAGATGATAGCCCCTCGGGAG
CTTCTTATGGCCAAGATTATGACCTTAGTCCCTCACGATCTCATACCT
CGAGCAATTATGACTCCTACAAGAAAAGTCCTGGAAGTACCTCGAGAA
GGCAGTCGGTCAGTCCCCCTTACAAGGAGCCTTCGGCCTACCAGTCC
AGCACCCGGTCACCGAGCCCCTACAGTAGGCGACAGAGATCTGTCA
GTCCCTATAGCAGGAGACGGTCGTCCAGCTACGAAAGAAGTGGCTCT
TACAGCGGGCGATCGCCCAGTCCCTATGGTCGAAGGCGGTCCAGCA
GCCCTTTCCTGAGCAAGCGGTCTCTGAGTCGGAGTCCACTCCCCAGT
AGGAAATCCATGAAGTCCAGAAGTAGAAGTCCTGCATATTCAAGACAT
TCATCTTCTCATAGTAAAAAGAAGAGATCCAGTTCACGCAGTCGTCAT
TCCAGTATCTCACCTGTCAGGCTTCCACTTAATTCCAGTCTGGGAGCT
GAACTCAGTAGGAAAAAGAAGGAAAGAGCAGCTGCTGCTGCTGCAGC
AAAGATGGATGGAAAGGAGTCCAAGGGTTCACCTGTATTTTTGCCTAG
AAAAGAGAACAGTTCAGTAGAGGCTAAGGATTCAGGTTTGGAGTCTAA
AAAGTTACCCAGAAGTGTAAAATTGGAAAAATCTGCCCCAGATACTGA
ACTGGTGAATGTAACACATCTAAACACAGAGGTAAAAAATTCTTCAGA
TACAGGGAAAGTAAAGTTGGATGAGAACTCCGAGAAGCATCTTGTTAA
AGATTTGAAAGCACAGGGAACAAGAGACTCTAAACCCATAGCACTGAA
AGAGGAGATTGTTACTCCAAAGGAGACAGAAACATCAGAAAAGGAGA
CCCCTCCACCTCTTCCCACAATTGCTTCTCCCCCACCCCCTCTACCAA
CTACTACCCCTCCACCTCAGACACCCCCTTTGCCACCTTTGCCTCCAA
TACCAGCTCTTCCACAGCAACCACCTCTGCCTCCTTCTCAGCCAGCAT
TTAGTCAGGTTCCTGCTTCCAGTACTTCAACTTTGCCCCCTTCTACTCA
CTCAAAGACATCTGCTGTGTCCTCTCAGGCAAATTCTCAGCCCCCTGT
ACAGGTTTCTGTGAAGACTCAAGTATCTGTAACAGCTGCTATTCCACA
CCTGAAAACTTCAACGTTGCCTCCTTTGCCCCTCCCACCCTTATTACC
TGGAGATGATGACATGGATAGTCCAAAAGAAACTCTTCCTTCAAAACC
TGTGAAGAAAGAGAAGGAACAGAGGACACGTCACTTACTCACAGACC
TTCCTCTCCCTCCAGAGCTCCCTGGTGGAGATCTGTCTCCCCCAGAC
TCTCCAGAACCAAAGGCAATCACACCACCTCAGCAACCATATAAAAAG
AGACCAAAAATTTGTTGTCCTCGTTATGGAGAAAGAAGACAAACAGAA
AGCGACTGGGGGAAACGCTGTGTGGACAAGTTTGACATTATTGGGAT
TATTGGAGAAGGAACCTATGGCCAAGTATATAAAGCCAAGGACAAAGA
CACAGGAGAACTAGTGGCTCTGAAGAAGGTGAGACTAGACAATGAGA
AAGAGGGCTTCCCAATCACAGCCATTCGTGAAATCAAAATCCTTCGTC
AGTTAATCCACCGAAGTGTTGTTAACATGAAGGAAATTGTCACAGATA
AACAAGATGCACTGGATTTCAAGAAGGACAAAGGTGCCTTTTACCTTG
TATTTGAGTATATGGACCATGACTTAATGGGACTGCTAGAATCTGGTT
TGGTGCACTTTTCTGAGGACCATATCAAGTCGTTCATGAAACAGCTAA
TGGAAGGATTGGAATACTGTCACAAAAAGAATTTCCTGCATCGGGATA
TTAAGTGTTCTAACATTTTGCTGAATAACAGTGGGCAAATCAAACTAGC
AGATTTTGGACTTGCTCGGCTCTATAACTCTGAAGAGAGTCGCCCTTA
CACAAACAAAGTCATTACTTTGTGGTACCGACCTCCAGAACTACTGCT
AGGAGAGGAACGTTACACACCAGCCATAGATGTTTGGAGCTGTGGAT
GTATTCTTGGGGAACTATTCACAAAGAAGCCTATTTTTCAAGCCAATCT
GGAACTGGCTCAGCTAGAACTGATCAGCCGACTTTGTGGTAGCCCTT
GTCCAGCTGTGTGGCCTGATGTTATCAAACTGCCCTACTTCAACACCA
TGAAACCGAAGAAGCAATATCGAAGGCGTCTACGAGAAGAATTCTCTT
TCATTCCTTCTGCAGCACTTGATTTATTGGACCACATGCTGACACTAG
ATCCTAGTAAGCGGTGCACAGCTGAACAGACCCTACAGAGCGACTTC
CTTAAAGATGTCGAACTCAGCAAAATGGCTCCTCCAGACCTCCCCCAC
TGGCAGGATTGCCATGAGTTGTGGAGTAAGAAACGGCGACGTCAGCG
ACAAAGTGGTGTTGTAGTCGAAGAGCCACCTCCATCCAAAACTTCTCG
AAAAGAAACTACCTCAGGGACAAGTACTGAGCCTGTGAAGAACAGCA
GCCCAGCACCACCTCAGCCTGCTCCTGGCAAGGTGGAGTCTGGGGC
TGGGGATGCAATAGGCCTTGCTGACATCACACAACAGCTGAATCAAA
GTGAATTGGCAGTGTTATTAAACCTGCTGCAGAGCCAAACCGACCTG
AGCATCCCTCAAATGGCACAGCTGCTTAACATCCACTCCAACCCAGA
GATGCAGCAGCAGCTGGAAGCCCTGAACCAATCCATCAGTGCCCTGA
CGGAAGCTACTTCCCAGCAGCAGGACTCAGAGACCATGGCCCCAGA
GGAGTCTTTGAAGGAAGCACCCTCTGCCCCAGTGATCCTGCCTTCAG
CAGAACAGACGACCCTTGAAGCTTCAAGCACACCAGCTGACATGCAG
AATATATTGGCAGTTCTCTTGAGTCAGCTGATGAAAACCCAAGAGCCA
GCAGGCAGTCTGGAGGAAAACAACAGTGACAAGAACAGTGGGCCAC
AGGGGCCCCGAAGAACTCCCACAATGCCACAGGAGGAGGCAGCAGC
ATGTCCTCCTCACATTCTTCCACCAGAGAAGAGGCCCCCTGAGCCCC
CCGGACCTCCACCGCCGCCACCTCCACCCCCTCTGGTTGAAGGCGA
TCTTTCCAGCGCCCCCCAGGAGTTGAACCCAGCCGTGACAGCCGCCT
TGCTGCAACTTTTATCCCAGCCTGAAGCAGAGCCTCCTGGCCACCTG
CCACATGAGCACCAGGCCTTGAGACCAATGGAGTACTCCACCCGACC
CCGTCCAAACAGGACTTATGGAAACACTGATGGGCCTGAAACAGGGT
TCAGTGCCATTGACACTGATGAACGAAACTCTGGTCCAGCCTTGACA
GAATCCTTGGTCCAGACCCTGGTGAAGAACAGGACCTTCTCAGGCTC
TCTGAGCCACCTTGGGGAGTCCAGCAGTTACCAGGGCACAGGGTCA
GTGCAGTTTCCAGGGGACCAGGACCTCCGTTTTGCCAGGGTCCCCTT
AGCGTTACACCCGGTGGTCGGGCAACCATTCCTGAAGGCTGAGGGA
AGCAGCAATTCTGTGGTACATGCAGAGACCAAATTGCAAAACTATGGG
GAGCTGGGGCCAGGAACCACTGGGGCCAGCAGCTCAGGAGCAGGC
CTTCACTGGGGGGGCCCAACTCAGTCTTCTGCTTATGGAAAACTCTAT
CGGGGGCCTACAAGAGTCCCACCAAGAGGGGGAAGAGGGAGAGGA
GTTCCTTACTAACCCAGAGACTTCAGTGTCCTGAAAGATTCCTTTCCT
ATCCATCCTTCCATCCAGTTCTCTGAATCTTTAATGAAATCATTTGCCA
GAGCGAGGTAATCATCTGCATTTGGCTACTGCAAAGCTGTCCGTTGTA
TTCCTTGCTCACTTGCTACTAGCAGGCGACTTACGAAATAATGATGTT
GGCACCAGTTCCCCCTGGATGGGCTATAGCCAGAACATTTACTTCAA
CTCTACCTTAGTAGATACAAGTAGAGAATATGGAGAGGATCATTACAT
TGAAAAGTAAATGTTTTATTAGTTCATTGCCTGCACTTACTGATCGGAA
GAGAGAAAGAACAGTTTCAGTATTGAGATGGCTCAGGAGAGGCTCTT
TGATTTTTAAAGTTTTGGGGTGGGGGATTGTGTGTGGTTTCTTTCTTTT
GAATTTTAATTTAGGTGTTTTGGGTTTTTTTCCTTTAAAGAGAATAGTGT
TCACAAAATTTGAGCTGCTCTTTGGCTTTTGCTATAAGGGAAACAGAG
TGGCCTGGCTGATTTGAATAAATGTTTCTTTCCTCTCCACCATCTCACA
TTTTGCTTTTAAGTGAACACTTTTTCCCCATTGAGCATCTTGAACATAC
TTTTTTTCCAAATAAATTACTCATCCTTAAAGTTTACTCCACTTTGACAA
AAGATACGCCCTTCTCCCTGCACATAAAGCAGGTTGTAGAACGTGGC
ATTCTTGGGCAAGTAGGTAGACTTTACCCAGTCTCTTTCCTTTTTTGCT
GATGTGTGCTCTCTCTCTCTCTTTCTCTCTCTCTCTCTCTCTCTCTCTC
TCTCTCTCTCTCTCTGTCTCGCTTGCTCGCTCTCGCTGTTTCTCTCTCT
TTGAGGCATTTGTTTGGAAAAAATCGTTGAGATGCCCAAGAACCTGGG
ATAATTCTTTACTTTTTTTGAAATAAAGGAAAGGAAATTCAGACTCTTAC
ATTGTTCTCTGTAACTCTTCAATTCTAAAATGTTTTGTTTTTTAAACCAT
GTTCTGATGGGGAAGTTGATTTGTAAGTGTGGACAGCTTGGACATTGC
TGCTGAGCTGTGGTTAGAGATGATGCCTCCATTCCTAGAGGGCTAATA
ACAGCATTTAGCATATTGTTTACACATATATTTTTATGTCAAAAAAAAAA
CAAAAACCTTTCAAACAGAGCATTGTGATATTGTCAAAGAGAAAAACA
AATCCTGAAGATACATGGAAATGTAACCTAGTTTAGGGTGGGTATTTT
TCTGAAGATACATCAATACCTGACCTTTTTTAAAAAAATAATTTTAAAAC
AGCATACTGTGAGGAAGAACAGTATTGACATACCCACATCCCAGCATG
TGTACCCTGCCAGTTCTTTTAGGGATTTTTCCTCCAAAGAGATTTGGAT
TTGGTTTTGGTAAAAGGGGTTAAATTGTGCTTCCAGGCAAGAACTTTG
CCTTATCATAAACAGGAAATGAAAAAGGGAAGGGCTGTCAGGATGGG
ATAATTTGGGAGGCTTCTCATTCTGGCTTCTATTTCTATGTGAGTACCA
GCATATAGAGTGTTTTAAAAACAGATACATGTCATATAATTTATCTGCA
CAGACTTAGACCTTCAGGAAACATAGGTTAAGCCCCCTTTTACAAAGA
AAAAGTAAACATACTTCAGCATCTTGGAGGGTAGTTTTCAAAACTCAA
GTTTCATGTTTCAATGCCAAGTTCTTATTTTAAAAAATAAAATCTACTTA
TAAGAGAAAGGTGCATTACTTAAAAAAAAAAAACTTTAAAGAAATGAAA
GAAGAACCCTCTTCAGATACTTACTTGAAGACTGTTTTCCCCTGTTAAT
GAGATATAGCTAGATATCGGTGTGTGTATTTCTTTATTATTCTCTGGTT
TTTGATCTGGCCTTGCCTCCAGGGCCAAACACTGATTTAGAAAGAGAG
CCTTCTAGCTATTTTGGCATTGATGGCTTTTTATACCAGTGTGTCCAGT
TAGATTTACTAGGCTTACTGACATGCTATTGGTAAATCGCATTAAAGTT
CATCTGAACCTTCTGTCTGTTGACTTCTTAGTCCTCAGACATGGGCCT
TTGTGTTTTAGAATATTTGAATTTGAGTTATTGGGCCCCACTCCCTGTT
TTTTATTAAAGAACGTGAGCCTGGGATACTTTCAGAAGTATCTGTTCAA
TGAAAAAAAGTTGGTTTCCCATCAAATATGAATAAAATTCTCTATATATT
TCATTGTATTTTGGTTATCAGCAGTCATCAATAATGTTTTTCCCTCCCC
TCTCCCACCTCTTATTTTTAATTATGCCAAATATCCTAAATAATATACTT
AAGCCTCCATTCCCTCATCCCTACTAGGGAAGGGGGTGAGTGTATGT
GTGAGTGTATGTGTATGTATGATCCCATCTCACCCCCACCCCCATTTT
GGGAGTCTTTTAAAATGAAAACAAAGTTTGGTAGTTTTGACTATTTCTA
AAAGCAGAGGAGAAAAAAAAACTTATTTAAATATCCTGGAATCTGTATG
GAGGAAGAAAAGGTATTTGTTAATTTTTCAGTTACGTTATCTATAAACA
TGATGGAAGTAAAGGTTTGGCAGAATTTCACCTTGACTATTTGAAAATT
ACAGACCCAATTAATTCCATTCAAAAGTGGTTTTCGTTTTGTTTTAATTA
TTGTACAATGAGAGATATTGTCTATTAAATACATTATTTTGAACAGATG
AGAAATCTGATTCTGTTCATGAGTGGGAGGCAAAACTGGTTTGACCGT
GATCATTTTTGTGGTTTTGAAAACAAATATACTTGACCCAGTTTCCTTA
GTTTTTTCTTCAACTGTCCATAGGAACGATAAGTATTTGAAAGCAACAT
CAAATCTATACGTTTAAAGCAGGGCAGTTAGCACAAATTTGCAAGTAG
AACTTCTATTAGCTTATGCCATAGACATCACCCAACCACTTGTATGTGT
GTGTGTATATATAATATGCATATATAGTTACCGTGCTAAAATGGTTACC
AGCAGGTTTTGAGAGAGAATGCTGCATCAGAAAAGTGTCAGTTGCCA
CCTCATTCTCCCTGATTTAGGTTCCTGACACTGATTCCTTTCTCTCTCG
TTTTTGACCCCCATTGGGTGTATCTTGTCTATGTACAGATATTTTGTAA
TATATTAAATTTTTTTCTTTCAGTTTATAAAAATGGAAAGTGGAGATTGG
AAAATTAAATATTTCCTGTTACTATACCAAAAAAAAAAAAAAAAAAAAA
SEQ ID NO. 10:
GGCGGCGGCTGGAGGAGAGCGCGGTGGAGAGCCGAGCGGGCGGG
CGGCGGGTGCGGAGCGGGCGAGGGAGCGCGCGCGGCCGCCACAAA
GCTCGGGCGCCGCGGGGCTGCATGCGGCGTACCTGGCCCGGCGCG
GCGACTGCTCTCCGGGCTGGCGGGGGCCGGCCGCGAGCCCCGGGG
GCCCCGAGGCCGCAGCTTGCCTGCGCGCTCTGAGCCTTCGCAACTC
GCGAGCAAAGTTTGGTGGAGGCAACGCCAAGCCTGAGTCCTTTCTTC
CTCTCGTTCCCCAAATCCGAGGGCAGCCCGCGGGCGTCATGCCCGC
GCTCCTCCGCAGCCTGGGGTACGCGTGAAGCCCGGGAGGCTTGGCG
CCGGCGAAGACCCAAGGACCACTCTTCTGCGTTTGGAGTTGCTCCCC
GCAACCCCGGGCTCGTCGCTTTCTCCATCCCGACCCACGCGGGGCG
CGGGGACAACACAGGTCGCGGAGGAGCGTTGCCATTCAAGTGACTG
CAGCAGCAGCGGCAGCGCCTCGGTTCCTGAGCCCACCGCAGGCTGA
AGGCATTGCGCGTAGTCCATGCCCGTAGAGGAAGTGTGCAGATGGG
ATTAACGTCCACATGGAGATATGGAAGAGGACCGGGGATTGGTACCG
TAACCATGGTCAGCTGGGGTCGTTTCATCTGCCTGGTCGTGGTCACC
ATGGCAACCTTGTCCCTGGCCCGGCCCTCCTTCAGTTTAGTTGAGGA
TACCACATTAGAGCCAGAAGAGCCACCAACCAAATACCAAATCTCTCA
ACCAGAAGTGTACGTGGCTGCGCCAGGGGAGTCGCTAGAGGTGCGC
TGCCTGTTGAAAGATGCCGCCGTGATCAGTTGGACTAAGGATGGGGT
GCACTTGGGGCCCAACAATAGGACAGTGCTTATTGGGGAGTACTTGC
AGATAAAGGGCGCCACGCCTAGAGACTCCGGCCTCTATGCTTGTACT
GCCAGTAGGACTGTAGACAGTGAAACTTGGTACTTCATGGTGAATGTC
ACAGATGCCATCTCATCCGGAGATGATGAGGATGACACCGATGGTGC
GGAAGATTTTGTCAGTGAGAACAGTAACAACAAGAGAGCACCATACTG
GACCAACACAGAAAAGATGGAAAAGCGGCTCCATGCTGTGCCTGCGG
CCAACACTGTCAAGTTTCGCTGCCCAGCCGGGGGGAACCCAATGCCA
ACCATGCGGTGGCTGAAAAACGGGAAGGAGTTTAAGCAGGAGCATCG
CATTGGAGGCTACAAGGTACGAAACCAGCACTGGAGCCTCATTATGG
AAAGTGTGGTCCCATCTGACAAGGGAAATTATACCTGTGTAGTGGAGA
ATGAATACGGGTCCATCAATCACACGTACCACCTGGATGTTGTGGAG
CGATCGCCTCACCGGCCCATCCTCCAAGCCGGACTGCCGGCAAATG
CCTCCACAGTGGTCGGAGGAGACGTAGAGTTTGTCTGCAAGGTTTAC
AGTGATGCCCAGCCCCACATCCAGTGGATCAAGCACGTGGAAAAGAA
CGGCAGTAAATACGGGCCCGACGGGCTGCCCTACCTCAAGGTTCTCA
AGGCCGCCGGTGTTAACACCACGGACAAAGAGATTGAGGTTCTCTAT
ATTCGGAATGTAACTTTTGAGGACGCTGGGGAATATACGTGCTTGGC
GGGTAATTCTATTGGGATATCCTTTCACTCTGCATGGTTGACAGTTCT
GCCAGCGCCTGGAAGAGAAAAGGAGATTACAGCTTCCCCAGACTACC
TGGAGATAGCCATTTACTGCATAGGGGTCTTCTTAATCGCCTGTATGG
TGGTAACAGTCATCCTGTGCCGAATGAAGAACACGACCAAGAAGCCA
GACTTCAGCAGCCAGCCGGCTGTGCACAAGCTGACCAAACGTATCCC
CCTGCGGAGACAGGTAACAGTTTCGGCTGAGTCCAGCTCCTCCATGA
ACTCCAACACCCCGCTGGTGAGGATAACAACACGCCTCTCTTCAACG
GCAGACACCCCCATGCTGGCAGGGGTCTCCGAGTATGAACTTCCAGA
GGACCCAAAATGGGAGTTTCCAAGAGATAAGCTGACACTGGGCAAGC
CCCTGGGAGAAGGTTGCTTTGGGCAAGTGGTCATGGCGGAAGCAGT
GGGAATTGACAAAGACAAGCCCAAGGAGGCGGTCACCGTGGCCGTG
AAGATGTTGAAAGATGATGCCACAGAGAAAGACCTTTCTGATCTGGTG
TCAGAGATGGAGATGATGAAGATGATTGGGAAACACAAGAATATCATA
AATCTTCTTGGAGCCTGCACACAGGATGGGCCTCTCTATGTCATAGTT
GAGTATGCCTCTAAAGGCAACCTCCGAGAATACCTCCGAGCCCGGAG
GCCACCCGGGATGGAGTACTCCTATGACATTAACCGTGTTCCTGAGG
AGCAGATGACCTTCAAGGACTTGGTGTCATGCACCTACCAGCTGGCC
AGAGGCATGGAGTACTTGGCTTCCCAAAAATGTATTCATCGAGATTTA
GCAGCCAGAAATGTTTTGGTAACAGAAAACAATGTGATGAAAATAGCA
GACTTTGGACTCGCCAGAGATATCAACAATATAGACTATTACAAAAAG
ACCACCAATGGGCGGCTTCCAGTCAAGTGGATGGCTCCAGAAGCCCT
GTTTGATAGAGTATACACTCATCAGAGTGATGTCTGGTCCTTCGGGGT
GTTAATGTGGGAGATCTTCACTTTAGGGGGCTCGCCCTACCCAGGGA
TTCCCGTGGAGGAACTTTTTAAGCTGCTGAAGGAAGGACACAGAATG
GATAAGCCAGCCAACTGCACCAACGAACTGTACATGATGATGAGGGA
CTGTTGGCATGCAGTGCCCTCCCAGAGACCAACGTTCAAGCAGTTGG
TAGAAGACTTGGATCGAATTCTCACTCTCACAACCAATGAGGAATACT
TGGACCTCAGCCAACCTCTCGAACAGTATTCACCTAGTTACCCTGACA
CAAGAAGTTCTTGTTCTTCAGGAGATGATTCTGTTTTTTCTCCAGACCC
CATGCCTTACGAACCATGCCTTCCTCAGTATCCACACATAAACGGCAG
TGTTAAAACATGAATGACTGTGTCTGCCTGTCCCCAAACAGGACAGCA
CTGGGAACCTAGCTACACTGAGCAGGGAGACCATGCCTCCCAGAGCT
TGTTGTCTCCACTTGTATATATGGATCAGAGGAGTAAATAATTGGAAAA
GTAATCAGCATATGTGTAAAGATTTATACAGTTGAAAACTTGTAATCTT
CCCCAGGAGGAGAAGAAGGTTTCTGGAGCAGTGGACTGCCACAAGC
CACCATGTAACCCCTCTCACCTGCCGTGCGTACTGGCTGTGGACCAG
TAGGACTCAAGGTGGACGTGCGTTCTGCCTTCCTTGTTAATTTTGTAA
TAATTGGAGAAGATTTATGTCAGCACACACTTACAGAGCACAAATGCA
GTATATAGGTGCTGGATGTATGTAAATATATTCAAATTATGTATAAATA
TATATTATATATTTACAAGGAGTTATTTTTTGTATTGATTTTAAATGGAT
GTCCCAATGCACCTAGAAAATTGGTCTCTCTTTTTTTAATAGCTATTTG
CTAAATGCTGTTCTTACACATAATTTCTTAATTTTCACCGAGCAGAGGT
GGAAAAATACTTTTGCTTTCAGGGAAAATGGTATAACGTTAATTTATTA
ATAAATTGGTAATATACAAAACAATTAATCATTTATAGTTTTTTTTGTAA
TTTAAGTGGCATTTCTATGCAGGCAGCACAGCAGACTAGTTAATCTAT
TGCTTGGACTTAACTAGTTATCAGATCCTTTGAAAAGAGAATATTTACA
ATATATGACTAATTTGGGGAAAATGAAGTTTTGATTTATTTGTGTTTAAA
TGCTGCTGTCAGACGATTGTTCTTAGACCTCCTAAATGCCCCATATTA
AAAGAACTCATTCATAGGAAGGTGTTTCATTTTGGTGTGCAACCCTGT
CATTACGTCAACGCAACGTCTAACTGGACTTCCCAAGATAAATGGTAC
CAGCGTCCTCTTAAAAGATGCCTTAATCCATTCCTTGAGGACAGACCT
TAGTTGAAATGATAGCAGAATGTGCTTCTCTCTGGCAGCTGGCCTTCT
GCTTCTGAGTTGCACATTAATCAGATTAGCCTGTATTCTCTTCAGTGAA
TTTTGATAATGGCTTCCAGACTCTTTGGCGTTGGAGACGCCTGTTAGG
ATCTTCAAGTCCCATCATAGAAAATTGAAACACAGAGTTGTTCTGCTG
ATAGTTTTGGGGATACGTCCATCTTTTTAAGGGATTGCTTTCATCTAAT
TCTGGCAGGACCTCACCAAAAGATCCAGCCTCATACCTACATCAGACA
AAATATCGCCGTTGTTCCTTCTGTACTAAAGTATTGTGTTTTGCTTTGG
AAACACCCACTCACTTTGCAATAGCCGTGCAAGATGAATGCAGATTAC
ACTGATCTTATGTGTTACAAAATTGGAGAAAGTATTTAATAAAACCTGT
TAATTTTTATACTGACAATAAAAATGTTTCTACAGATATTAATGTTAACA
AGACAAAATAAATGTCACGCAACTTATTTTTTTAATAAAAAAAAAAAAAA
A
SEQ ID NO. 11:
GGCGGCGGCTGGAGGAGAGCGCGGTGGAGAGCCGAGCGGGCGGG
CGGCGGGTGCGGAGCGGGCGAGGGAGCGCGCGCGGCCGCCACAAA
GCTCGGGCGCCGCGGGGCTGCATGCGGCGTACCTGGCCCGGCGCG
GCGACTGCTCTCCGGGCTGGCGGGGGCCGGCCGCGAGCCCCGGGG
GCCCCGAGGCCGCAGCTTGCCTGCGCGCTCTGAGCCTTCGCAACTC
GCGAGCAAAGTTTGGTGGAGGCAACGCCAAGCCTGAGTCCTTTCTTC
CTCTCGTTCCCCAAATCCGAGGGCAGCCCGCGGGCGTCATGCCCGC
GCTCCTCCGCAGCCTGGGGTACGCGTGAAGCCCGGGAGGCTTGGCG
CCGGCGAAGACCCAAGGACCACTCTTCTGCGTTTGGAGTTGCTCCCC
GCAACCCCGGGCTCGTCGCTTTCTCCATCCCGACCCACGCGGGGCG
CGGGGACAACACAGGTCGCGGAGGAGCGTTGCCATTCAAGTGACTG
CAGCAGCAGCGGCAGCGCCTCGGTTCCTGAGCCCACCGCAGGCTGA
AGGCATTGCGCGTAGTCCATGCCCGTAGAGGAAGTGTGCAGATGGG
ATTAACGTCCACATGGAGATATGGAAGAGGACCGGGGATTGGTACCG
TAACCATGGTCAGCTGGGGTCGTTTCATCTGCCTGGTCGTGGTCACC
ATGGCAACCTTGTCCCTGGCCCGGCCCTCCTTCAGTTTAGTTGAGGA
TACCACATTAGAGCCAGAAGAGCCACCAACCAAATACCAAATCTCTCA
ACCAGAAGTGTACGTGGCTGCGCCAGGGGAGTCGCTAGAGGTGCGC
TGCCTGTTGAAAGATGCCGCCGTGATCAGTTGGACTAAGGATGGGGT
GCACTTGGGGCCCAACAATAGGACAGTGCTTATTGGGGAGTACTTGC
AGATAAAGGGCGCCACGCCTAGAGACTCCGGCCTCTATGCTTGTACT
GCCAGTAGGACTGTAGACAGTGAAACTTGGTACTTCATGGTGAATGTC
ACAGATGCCATCTCATCCGGAGATGATGAGGATGACACCGATGGTGC
GGAAGATTTTGTCAGTGAGAACAGTAACAACAAGAGAGCACCATACTG
GACCAACACAGAAAAGATGGAAAAGCGGCTCCATGCTGTGCCTGCGG
CCAACACTGTCAAGTTTCGCTGCCCAGCCGGGGGGAACCCAATGCCA
ACCATGCGGTGGCTGAAAAACGGGAAGGAGTTTAAGCAGGAGCATCG
CATTGGAGGCTACAAGGTACGAAACCAGCACTGGAGCCTCATTATGG
AAAGTGTGGTCCCATCTGACAAGGGAAATTATACCTGTGTAGTGGAGA
ATGAATACGGGTCCATCAATCACACGTACCACCTGGATGTTGTGGAG
CGATCGCCTCACCGGCCCATCCTCCAAGCCGGACTGCCGGCAAATG
CCTCCACAGTGGTCGGAGGAGACGTAGAGTTTGTCTGCAAGGTTTAC
AGTGATGCCCAGCCCCACATCCAGTGGATCAAGCACGTGGAAAAGAA
CGGCAGTAAATACGGGCCCGACGGGCTGCCCTACCTCAAGGTTCTCA
AGCACTCGGGGATAAATAGTTCCAATGCAGAAGTGCTGGCTCTGTTC
AATGTGACCGAGGCGGATGCTGGGGAATATATATGTAAGGTCTCCAA
TTATATAGGGCAGGCCAACCAGTCTGCCTGGCTCACTGTCCTGCCAA
AACAGCAAGCGCCTGGAAGAGAAAAGGAGATTACAGCTTCCCCAGAC
TACCTGGAGATAGCCATTTACTGCATAGGGGTCTTCTTAATCGCCTGT
ATGGTGGTAACAGTCATCCTGTGCCGAATGAAGAACACGACCAAGAA
GCCAGACTTCAGCAGCCAGCCGGCTGTGCACAAGCTGACCAAACGTA
TCCCCCTGCGGAGACAGGTAACAGTTTCGGCTGAGTCCAGCTCCTCC
ATGAACTCCAACACCCCGCTGGTGAGGATAACAACACGCCTCTCTTC
AACGGCAGACACCCCCATGCTGGCAGGGGTCTCCGAGTATGAACTTC
CAGAGGACCCAAAATGGGAGTTTCCAAGAGATAAGCTGACACTGGGC
AAGCCCCTGGGAGAAGGTTGCTTTGGGCAAGTGGTCATGGCGGAAG
CAGTGGGAATTGACAAAGACAAGCCCAAGGAGGCGGTCACCGTGGC
CGTGAAGATGTTGAAAGATGATGCCACAGAGAAAGACCTTTCTGATCT
GGTGTCAGAGATGGAGATGATGAAGATGATTGGGAAACACAAGAATA
TCATAAATCTTCTTGGAGCCTGCACACAGGATGGGCCTCTCTATGTCA
TAGTTGAGTATGCCTCTAAAGGCAACCTCCGAGAATACCTCCGAGCC
CGGAGGCCACCCGGGATGGAGTACTCCTATGACATTAACCGTGTTCC
TGAGGAGCAGATGACCTTCAAGGACTTGGTGTCATGCACCTACCAGC
TGGCCAGAGGCATGGAGTACTTGGCTTCCCAAAAATGTATTCATCGA
GATTTAGCAGCCAGAAATGTTTTGGTAACAGAAAACAATGTGATGAAA
ATAGCAGACTTTGGACTCGCCAGAGATATCAACAATATAGACTATTAC
AAAAAGACCACCAATGGGCGGCTTCCAGTCAAGTGGATGGCTCCAGA
AGCCCTGTTTGATAGAGTATACACTCATCAGAGTGATGTCTGGTCCTT
CGGGGTGTTAATGTGGGAGATCTTCACTTTAGGGGGCTCGCCCTACC
CAGGGATTCCCGTGGAGGAACTTTTTAAGCTGCTGAAGGAAGGACAC
AGAATGGATAAGCCAGCCAACTGCACCAACGAACTGTACATGATGAT
GAGGGACTGTTGGCATGCAGTGCCCTCCCAGAGACCAACGTTCAAGC
AGTTGGTAGAAGACTTGGATCGAATTCTCACTCTCACAACCAATGAGG
AATACTTGGACCTCAGCCAACCTCTCGAACAGTATTCACCTAGTTACC
CTGACACAAGAAGTTCTTGTTCTTCAGGAGATGATTCTGTTTTTTCTCC
AGACCCCATGCCTTACGAACCATGCCTTCCTCAGTATCCACACATAAA
CGGCAGTGTTAAAACATGAATGACTGTGTCTGCCTGTCCCCAAACAG
GACAGCACTGGGAACCTAGCTACACTGAGCAGGGAGACCATGCCTCC
CAGAGCTTGTTGTCTCCACTTGTATATATGGATCAGAGGAGTAAATAA
TTGGAAAAGTAATCAGCATATGTGTAAAGATTTATACAGTTGAAAACTT
GTAATCTTCCCCAGGAGGAGAAGAAGGTTTCTGGAGCAGTGGACTGC
CACAAGCCACCATGTAACCCCTCTCACCTGCCGTGCGTACTGGCTGT
GGACCAGTAGGACTCAAGGTGGACGTGCGTTCTGCCTTCCTTGTTAA
TTTTGTAATAATTGGAGAAGATTTATGTCAGCACACACTTACAGAGCAC
AAATGCAGTATATAGGTGCTGGATGTATGTAAATATATTCAAATTATGT
ATAAATATATATTATATATTTACAAGGAGTTATTTTTTGTATTGATTTTA
AATGGATGTCCCAATGCACCTAGAAAATTGGTCTCTCTTTTTTTAATAGC
TATTTGCTAAATGCTGTTCTTACACATAATTTCTTAATTTTCACCGAGCA
GAGGTGGAAAAATACTTTTGCTTTCAGGGAAAATGGTATAACGTTAAT
TTATTAATAAATTGGTAATATACAAAACAATTAATCATTTATAGTTTTTT
TTGTAATTTAAGTGGCATTTCTATGCAGGCAGCACAGCAGACTAGTTAA
TCTATTGCTTGGACTTAACTAGTTATCAGATCCTTTGAAAAGAGAATAT
TTACAATATATGACTAATTTGGGGAAAATGAAGTTTTGATTTATTTGTG
TTTAAATGCTGCTGTCAGACGATTGTTCTTAGACCTCCTAAATGCCCC
ATATTAAAAGAACTCATTCATAGGAAGGTGTTTCATTTTGGTGTGCAAC
CCTGTCATTACGTCAACGCAACGTCTAACTGGACTTCCCAAGATAAAT
GGTACCAGCGTCCTCTTAAAAGATGCCTTAATCCATTCCTTGAGGACA
GACCTTAGTTGAAATGATAGCAGAATGTGCTTCTCTCTGGCAGCTGGC
CTTCTGCTTCTGAGTTGCACATTAATCAGATTAGCCTGTATTCTCTTCA
GTGAATTTTGATAATGGCTTCCAGACTCTTTGGCGTTGGAGACGCCTG
TTAGGATCTTCAAGTCCCATCATAGAAAATTGAAACACAGAGTTGTTCT
GCTGATAGTTTTGGGGATACGTCCATCTTTTTAAGGGATTGCTTTCAT
CTAATTCTGGCAGGACCTCACCAAAAGATCCAGCCTCATACCTACATC
AGACAAAATATCGCCGTTGTTCCTTCTGTACTAAAGTATTGTGTTTTGC
TTTGGAAACACCCACTCACTTTGCAATAGCCGTGCAAGATGAATGCAG
ATTACACTGATCTTATGTGTTACAAAATTGGAGAAAGTATTTAATAAAA
CCTGTTAATTTTTATACTGACAATAAAAATGTTTCTACAGATATTAATGT
TAACAAGACAAAATAAATGTCACGCAACTTATTTTTTTAATAAAAAAAAA
AAAAAA
SEQ ID NO. 12:
GTGATGGCCTCCCTGAAATTAAACATTTCTATTAGTGGCTTCCCGTTA
ATCTCATCCTTCTTAGATCAAACCTCGTTATATCTCCTGCCTATCTCTT
TTGCATTCCAAAGTTCAGTTTTATTAAATCCCAGGGTCTAAGATTTTTT
CTTTGAGAATTTATCTCCAGTGTTTCTATGGAAATTAAAAAAGAAAATT
AGGATAATTCAATGTCGAAATGTTGCATGCATCTTTTGAGAAATTTATA
TTTTGTAGGTTGAAGGACTTGCTTTTTGGGCAGCGTATTTTTGGAGGT
GGAATGTAGTTATTTTAATAACCATGTCCTAATTATTTATAGCTTCCTG
CCTGACACAGCTCACTTCAAGAAGTGCACAATGTCAGAACGTGGAATT
AAGTGGGCTTGTGAATATTGTACGTATGAAAACTGGCCATCTGCAATC
AAGTGTACTATGTGTCGTGCCCAAAGACCTAGTGGAACAATTATTACA
GAAGATCCATTTAAAAGTGGTTCAAGTGATGTTGGTAGAGATTGGGAT
CCTTCCAGCACCGAAGGAGGAAGTAGTCCTTTGATATGTCCAGACTCT
AGTGCAAGACCAAGGGTGAAATCTTCGTATAGCATGGAAAATGCAAAT
AAGTGGTCATGCCACATGTGTACATATTTGAACTGGCCAAGAGCAATC
AGATGTACCCAGTGCTTATCCCAACGTAGGACCAGGAGTCCTACAGA
ATCTCCTCAGTCCTCAGGATCTGGCTCAAGACCAGTTGCTTTTTCTGT
TGATCCTTGTGAGGAATACAATGATAGAAATAAACTGAACACTAGGAC
ACAGCACTGGACTTGCTCTGTTTGCACATATGAAAACTGGGCCAAGG
CTAAAAGATGTGTTGTTTGTGATCATCCCAGACCTAATAACATTGAAG
CAATAGAATTGGCAGAGACTGAAGAGGCTTCTTCAATAATAAATGAGC
AAGACAGAGCTCGATGGAGGGGAAGTTGCAGTAGTGGTAATAGCCAA
AGGAGATCACCTCCTGCTACGAAGCGGGACTCTGAAGTGAAAATGGA
TTTTCAGAGGATTGAATTGGCTGGTGCTGTGGGAAGCAAGGAGGAAC
TTGAAGTAGACTTTAAAAAACTAAAGCAAATTAAAAACAGGATGAAAAA
GACTGATTGGCTCTTCCTCAATGCTTGTGTGGGGGTTGTAGAAGGTG
ATTTAGCTGCCATAGAAGCATACAAGTCATCAGGAGGAGACATTGCAC
GTCAGCTCACCGCAGATGAAGTACGCTTGCTGAATCGTCCTTCTGCC
TTTGATGTTGGCTATACTCTTGTACACTTGGCTATACGTTTTCAGAGGC
AGGATATGCTAGCAATATTGCTTACAGAGGTGTCTCAACAAGCAGCAA
AGTGTATTCCAGCAATGGTGTGTCCTGAACTGACAGAACAAATCCGGA
GAGAGATAGCTGCCTCTCTTCATCAGAGAAAGGGGGATTTTGCTTGCT
ATTTTCTGACTGACCTTGTAACATTTACATTGCCAGCAGATATTGAAGA
TTTGCCCCCAACAGTCCAAGAAAAATTATTTGATGAGGTGCTTGATAG
AGACGTTCAAAAAGAATTAGAAGAAGAATCTCCAATTATTAACTGGTC
CTTGGAATTGGCTACACGTTTGGACAGTCGACTGTATGCACTTTGGAA
CCGGACTGCAGGAGACTGCCTACTTGATTCAGTTCTACAAGCTACCT
GGGGCATCTATGACAAGGACTCAGTGCTTCGGAAAGCCCTGCATGAC
AGCCTGCATGACTGTTCACATTGGTTTTACACACGCTGGAAAGATTGG
GAATCATGGTATTCTCAGAGCTTTGGTTTACATTTTTCCTTGAGAGAAG
AACAGTGGCAAGAAGACTGGGCATTTATACTCTCTCTTGCTAGTCAGC
CTGGAGCAAGCTTGGAGCAGACGCACATTTTTGTACTGGCACATATTC
TTAGACGACCAATTATAGTTTATGGAGTAAAATATTACAAGAGTTTCCG
GGGAGAAACTTTAGGATATACTCGGTTTCAAGGTGTTTATCTGCCTTT
GTTGTGGGAACAGAGTTTTTGTTGGAAAAGTCCGATTGCTCTGGGTTA
TACGAGGGGCCACTTCTCTGCTTTGGTTGCCATGGAAAATGATGGCT
ATGGCAACCGAGGTGCTGGTGCTAATCTCAATACCGATGATGATGTC
ACCATCACATTTTTGCCTCTGGTTGACAGTGAAAGGAAGCTACTCCAT
GTGCACTTCCTTTCTGCTCAGGAGCTAGGTAATGAGGAACAGCAAGA
AAAACTGCTCAGGGAGTGGCTGGACTGCTGTGTGACGGAGGGGGGA
GTTCTGGTTGCCATGCAGAAGAGTTCTCGGCGGCGAAATCACCCCCT
GGTCACTCAGATGGTAGAAAAATGGCTTGACCGCTACCGACAGATCC
GGCCGTGTACATCCCTGTCTGATGGAGAGGAAGATGAGGATGATGAA
GATGAATGAAAAAAAAAATCAAACAGCAGAAGACCAAGGCATCAGATC
TGTAATGACCCTAAAGTTAGTGTGGTGCTCCAAGCAGAGTCGACATCA
TGGAATGAACCAAATCTGGCAGGATCTGCTCGGGGAAGTGTTTTCCT
GGACCACACACACCTTATGGAGATAATGCCTCTGCTGCGTGAGGAGA
CAGAGAACTTTAGTTGGACTACAGTTTGTAAAAAAAACTAATTTTATTA
AGACAGAACTTTTTTTCCTTCCAAATTGTAAATCTGTCTATAAATGTAA
CGCATGTGGTTGTGTAAGACATTGTTTAATAGGAAAAGTTGTACCAGC
ATCTTCATATTATTGAGAAAATTTTTTCCAGCATGGGCACTTAGAAAAA
GCACATGGCAAATGGCTCTTTGTTCCTTTCAGATATTATTTCAGTAGAA
CCTGGCATTCTACTTTCACCTTAAAAGATCCATCTAAGTCTCAGATCTG
GAAACGTTTTGTACCGATTATCCACAGCAAAACAAAAATAAGCTTTTAT
TTTATTAATAATTTCGTTCCTCTTGTGCCCAATCAAATCTTTTAGGAACA
AACTGCAAGAAAAGCTAAGAATGTTTTAGAGTGAACTAAATACAGACA
TTGCTTACTTGTTTTGAAGAGGGTTTTGGTTTTGGTTATTGTGTCTTTA
AGTTTTCTGATATGCCCCCTTTCAATATTTAGATATTTATTTGTTGGGA
AGAATACCTTAAAATGAGGGTTCTTATTCCAGATTCTGGGCAGTGGTC
TGTGAGTAGTTTTTTTCCTGGATGAAAAGGGAGCAAGCCCACTTGTCA
CTAAATGAATTGTGTGAAATGTGCTCACTTGGACTCCATCAACAATGT
GCTGCTCCCAGATTGCCATGCCAGAGGGTCTTCGGATTCTTCCTTCTA
TCACCTCTGCTCTAAGCAAATCTTGTTAGAAGGGCATGCCTTTGCTTA
GGCAGATTGGGAATACCAATTCACTACAGAATAAAGATTTTAAAAATG
CAATAAGGTGGCAAATGCATTGTATGAAGAATTTCTCAGTGTTTAGTCT
GAGAATTTTTGCATGTTGGTTAATTGTGGCCATTCTTTAATTTAAAGTT
AAAACTATAATCTTAGGTAGAAAAACTTTTTTATAAGAAGTATTATTTGA
CCACTTCAGGTATACATTCAATACTGGGTAAAAATTTCAGACCTATCTC
AGGAACACAGAAATATTTGGTGTCCTGATAAGCACTTTCTAGACTATT
GATGTGGCCAGGAATTTGGAAAGACGACACACGCACGCGCGCGCGC
GCACACACACACACACACACACACACACACACAGTTTTTTCCTTCCCT
GTGATGAAAAAGGCTGTGAAAACCTTAAAGTATTTGCTTGCTTCTTGTT
TTGTTTAGTTGATAATGAAATGTGTACAACCTCAAATTTGCTGCCAGAA
TACTAAAAATAGAAAAATACCCACAAAACTGTCATGTCTTTAGTTCTTT
CCCCCCGAAAACTCAGTAAAAAGGTGTTCCCAGGATGAAAAGATCATT
TTTTGCTGCATGCTAAATCTTGCAGGAAAAATGATTTTTTAGTACGATT
CTGTAGAAATGAATCTTTGATATAATGTAAATGCTGCTGTTTGTTTCAA
GTGGTGAATGTGTTGTTAAAAATTGGCTGTTTGCTTTCATTTTGGCCAA
TAAGTAATCAAGTTTGTAGAAAATGTTAGCATTCTGACTACTTAGCATC
TGTAGTAATTTCTCTATGTATAGGGATAATTTTTTAGTGGGCAGAGATC
CTGTTCTAGTTGCCTGTTAAGCAAAATCTGCCCTCCCAATTGAAAAAG
CCAAAGAGAATTGTTAGAGGGAAAAGCATGTAGCCATTGCAGTCTGC
ATTGCAGCCAGCGTTGTCCAGAGTACACGCTCAGCACTTAGCTTCTAC
TGTGTGTTGTGGTCTGGTGAGTGTTGTTTCCCCTGAGCGCTCTATTAT
TTATTTATTTATTATCAATCAGTGACCCTGACCACATAGTGTGATAGGT
GCAGCATTCTTCCCTGTGGGAAAGAATTAAAGATGGTTCCATTTCCTA
GGCTACAGACAGGAATGGGGCTCTAAATGGTTTTCATAGACTGGCTG
TTAAAGGCCAAAAATTTTGGTAAATCAATGCTATATTATGCTCTTGAAC
TATTAAAACAGCCATAATTATTGTCCCAAGATAGAATATAGTCCTTTTT
CAAAGATGATTATACGTGGCTAGGTGACAGACATTAATGACTGACTCT
GGAGAGTAAGTCATACCTGCACTCTGTGGACTTGATGGTTCTTTTTCT
AGAGCAAACAGAGCGTGGCATTTTGTTTTGACTTGTTCTTCCTTGGGG
TCAAATTTATATATATATATATAAATTTTTGTTTGGGCGACCAAGATCTA
ATAATTAAAACCCAGGTGGACCATGGATTCA
SEQ ID NO. 17:
GCACCTTCAAAGGGACACCTACGGCAGAGAACCCAGAGTACCTGGGT
CTGGACGTGCCAGTGTGAACCAGAAGGCCAAGTCCGCAGAAGCCCT
GATGTGTCCTCAGGGAGCAGGGAAGGCCTGACTTCTGCTGGCATCAA
GAGGTGGGAGGGCCCTCCGACCACTTCCAGGGGAACCTGCCATGCC
AGGAACCTGTCCTAAGGAACCTTCCTTCCTGCTTGAGTTCCCAGATGG
CTGGAAGGGGTCCAGCCTCGTTGGAAGAGGAACAGCACTGGGGAGT
CTTTGTGGATTCTGAGGCCCTGCCCAATGAGACTCTAGGGTCCAGTG
GATGCCACAGCCCAGCTTGGCCCTTTCCTTCCAGATCCTGGGTACTG
AAAGCCTTAGGGAAGCTGGCCTGAGAGGGGAAGCGGCCCTAAGGGA
GTGTCTAAGAACAAAAGCGACCCATTCAGAGACTGTCCCTGAAACCTA
GTACTGCCCCCCATGAGGAAGGAACAGCAATGGTGTCAGTATCCAGG
CTTTGTACA
SEQ ID NO. 18:
CCCTCGGAGGCAGAGGAAGGAAAATGGGGATGGCTGGGGCTCTCTC
CATCCTCCTTTTCTCCTTGCCNTTCGCATGGCTGGCCTTCCCCTCCAA
AACCTCCATTCCCCTGCTGCCAGCCCCTTTGCCATAGCCTGATTTTGG
GGAGGAGGAAGGGGCGATTTGAGGGAGAAGGGGAGAAAGCTTATGG
CTGGGTCTGGTTTCTTNCCCTTCCCAGAGGGTCTTACTGTTCCAGGGT
GGCCCCAGGGCAGGCAGGGGCCACACTATNNCCTGNGCCCTNGTAA
AGGTGACCCCTNNNNNNNNNNNNNNNNNNNNNNGCATGTTCCTGCC
CCACAGGAATAGAATGGAGGGAGCTCCAGAAACTTTCCATCCCAAAG
GCAGTCTCCGTGGTTGAAGCAGACTGGATTTTTGCTCTGCCCCTGAC
CCCTTGTCCCTCTTTGAGGGAGGGGAGCTATGCTAGGACTCCAACCT
CAGGGACTCGGGTGGCCTGCGCTAGCTTCTTTTGATACTGAAAA
SEQ ID NO. 19:
AGCAGTATCCGGGCATCGAGATCGAGTCGCGCCTCGGGGGCACAGG
TGCCTTTGAGATAGAGATAAATGGACAGCTGGTGTTCTCCAAGCTGGA
GAATGGGGGCTTTCCCTATGAGAAAGATCTCATTGAGGCCATCCGAA
GAGCCAGTAATGGAGAAACCCTAGAAAAGATCACCAACAGCCGTCCT
CCCTGCGTCATCCTGTGACTGCACAGGACTCTGGGTTCCTGCTCTGT
TCTGGGGTCCAAACCTTGGTCTCCCTTTGGTCCTGCTGGGAGCTCCC
CCTGCCTCTTTCCCCTACTTAGCTCCTTAGCAAAGAGACCCTGGCCTC
CACTTTGCCCTTTGGGTACAAAGAAGGAATAGAAGATTCCGTGGCCTT
GGGGGCAGGAGAGAGACACTCTCCATGAACACTTCTCCAGCCACCTC
ATACCCCCTTCCCAGGGTAAGTGCCCACGAAAGCCCAGTCCACTCTT
CGCCTCGGTAATACCTGTCTGATGCCACAGATTTTATTTATTCTCCCCT
AACCCAGGGCAATGTCA
SEQ ID NO. 20:
AGAATTACCAGCAGGCACAGTCTCGCCATCTGCATCCATCTTGTTTGG
GCTCCCCACCCTTGAGAAGTGCCTCAGATAATACCCTGGTGGCCATG
GACTTCTCTGGCCATGCTGGGCGTGTCATTGAGAACCCCCGGGAGG
CTCTGAGTGTGGCCCTGGAGGAGGCCCAGGCCTGGAGGAAGAAGAC
AAACCACCGCCTCAGCCTGCCCATGCCAGCCTCCGGCACGAGCCTC
AGTGCAGCCTGTTCCTGGTCCGGGAGAGTCAGCGGAACCCCCAGGG
CTTTGTCCTCTCTTTGTGCCACCTGCAGAAAGTGAAGCATTATCTCAT
CCTGCCGAGCGAGGAGGAGGGTCGCCTGTACTTCAGCATGGATGA
SEQ ID NO. 21:
GCAGCCCCTCGGAGGCAGAGGAAGGAAAATGGGGATGGCTGGGGCT
CTCTCCATCCTCCTTTTCTCCTTGCCTTCGCATGGNCTGGCCTTCCCC
TCCAAAACCTCCATTCCCCTGCTGCCAGCCCCTTTGCCATAGCCTGAT
TTTGGGGAGGAGGAAGGGGCGATTTGAGGGAGAAGGGGAGAAAGCT
TATGGCTGGGTCTGGTTTCTTCCCTTCCCAGAGGGTCTTACTGTTCCA
GGGTGGCCCCAGGGCAGGCAGGGGCCACACTATGCCTGCGCCCTG
GTAAAGGTGACCCCTGCCATTTACCAGCAGCCCTGGCATGTTCCTGC
CCCACAGGAATAGAATGGAGGGAGCTCCAGAAACTTTCCATCCCAAA
GGCAGTCTCCGTGGTTGAAGCAGACTGGATTTTTGCTCTGCCCCTGA
CCCCTTGTCCCTCTTTGAGGGAGGGGAGCTATGCTAGGACTCCAACC
TCAGGGACTCGGGTGGCCTGCGCTAGCTTCTTTTGATACTGA
SEQ ID NO. 22:
GACAGCGCATCAGCGAGCTGGGGGCCCGGGCGTGACTGTGCCCCCT
CCCACCCTGCGGGCCAGGGTCCTGTCGCCACCACTTCCAGAGCCAG
AAAGGGTGCCAGTTGGGCTCGCACTGCCCACATGGGACCTGGCCCC
AGGCTGTCACCCTCCACCGAGCCACGCAGTGCCTGGAGTTGACTGAC
TGAGCAGGCTGTGGGGTGGAGCACTGGACTCCGGGGCCCCACTGGC
TGGAGGAAGTGGGGTCTGGCCTGTTGATGTTTACATGGCGCCCTGCC
TCCTGGAGGACCAGATTGCTCTGCCCCACCTTGCCAGGGCAGGGTCT
GGGCTGGGCACCTGACTTGGCTGGGGAGGACCAGGGCCCTGGGCA
GGGCAGGGCAGCCTGTCACCCGTGTGAAGATGAAGGGGCTCTTCAT
CTGCCTGCGCTCTCGTCGGTTTTTTTAGGATTATTGAAAGAGTCTGGG
ACCCTTGTTGGGGAGT
SEQ ID NO. 23:
CACTCGTGAGTCCAACGGTCTTTTCTGCAGAAAGGAGGACTTTCCTTT
CAGGGGTCTTTCTGGGGCTCTTACTATAAAAGGGGACCAACTCTCCC
TTTGTCATATCTTGTTTCTGATGACAAAA
SEQ ID NO. 24:
CCTGGTCACCTACAACACAGACACGTTTGAGTCCATGCCCAATCCCG
AGGGCCGGTATACATTCGGCGCCAGCTGTGTCACTGCCTGTCCCTAC
AACTACCTTTCTACGGACGTGGGATCCTGCACCCTCGTCTGCCCCCT
GCACAACCAAGAGGTGACAGCAGAGGATGGAACACAGCGGTGTGAG
AAGTGCAGCAAGCCCTGTGCC
SEQ ID NO. 25:
ATAATATACTTAAGCCTCCATTCCCTCATCCCTACTAGGGAAGGGGGT
GAGTGTATGTGTGAGTGTATGTGTATGTATGATCCCATCTCACCCCCA
CCCCCATTTTGGGAGTCTTTTAAAATGAAAACAAAGTTTGGTAGTTTTG
ACTATTTCTAAAAGCAGAGGAGAAAAAAAAACTTATTTAAATATCCTGG
AATCTGTATGGAGGAAGAAAAGGTATTTGTTAATTTTTCAGTTACGTTA
TCTATAAACATGATGGAAGTAAAGGTTTGGCAGAATTTCACCTTGACT
ATTTGAAAATTACAGACCCAATTAATTCCATTCAAAAGTGGTTTTCG
SEQ ID NO. 26:
GTGTTCACAAAATTTGAGCTGCTCTTTGGCTTTTGCTATAAGGGAAAC
AGAGTGGCCTGGCTGATTTGAATAAATGTTTCTTTCCTCTCCACCATC
TCACATTTTGCTTTTAAGTGAACACTTTTTCCCCATTGAGCATCTTGAA
CATACTTTTTTTCCAAATAAATTACTCATCCTTAAAGTTTACTCCACTTT
GACAAAAGATACGCCCTTCTCCCTGCACATAAAGCAGGTTGTAGAAC
GTGGCATTCTTGGGCAAGTAGGTAGACTTTACCCAGTCTCTTTCCTTT
TTTGCTGATGTGTGCTCTCTCTCTCTCTTTCTCTCTCTCTCTCTCTCTC
TCTCTCTCTCTGTCTGTCTCGCTTGCTCGCTCTCGCTGTTTCTCTCTCT
TTGAGGCATTTGTTTGGAAAAAATCGTTGAGATGCCCAAGAACCT
SEQ ID NO. 27:
GAAATCTGGTACTGCATGGACTGGAGGGCAGAGGAGTTAGATTCCAG
TGGTTTTCTAATTTGGTTTCTGACTTCTGCCAGCCCCCAACCCATTCCT
TTCTAAGATTCGATACTCTGGCTGGGCTCTGGCTGACTTCCAGCCTTC
TCAGATGGAGCCAGGATTACATCTGTGTCTTTGCATTTTGTATCCAGG
TTTCGGCTGAGTCCAGCTCCTCCATGAACTCCAACACCCCGCTGGTG
AGGATAACAACACGCCTCTCTTCAACGGCAGACACCCCCATGCTGGC
AGGGGTCTCCGAGTATGAACTTCCAGAGGACCCAAAATGGGAGTTTC
CAAGAGATAAGTGAGTACTTCTCTTGGCCATGTCCCAGGATGGAGAC
TCAGCTATAAATGGGGATATTGGATTAACATTTTCTTTTTATGACCCTT
AGCCACAAAGGTCTTGGTGTGATGATGTCAGCAG
SEQ ID NO. 28:
GGGAGGCAAAACTGGTTTGACCGTGATCATTTTTGTGGTTTTGAAAAC
AAATATACTTGACCCAGTTTCCTTAGTTTTTTCTTCAACTGTCCATAGG
AACGATAAGTATTTGAAAGCAACATCAAATCTATACGTTTAAAGCAGG
GCAGTTAGCACAAATTTGCAAGTAGAACTTCTATTAGCTTATGCCATA
GACATCACCCAACCACTTGTATGTGTGTGTGTATATATAATATGCATAT
ATAGTTACCGTGCTAAAATGGTTACCAGCAGGTTTTGAGAGAGAATGC
TGCATCAGAAAAGTGTCAGTTGCCACCTCATTCTCCCTGATTTAGGTT
CCTGACACTGATTCCTTTCTCTCTCGTTTTTGACCCCCATTGGGTGTAT
CTTGTCTA
SEQ ID NO. 29:
AGCATGTAGCCATTGCAGTCTGCATTGCAGCCAGCGTTGTCCAGAGA
GTACACGCTCAGCACTTAGCTTCTACTGTGTGTTGTGGTCTGGTGAGT
GTTGTTTCCCCTGAGCGCTCTATTNATTTATTTATTTATTATCAATCAGT
GACCCTGACCACATAGTGTGATAGGTGCAGCATTCTTCCCTGTGGGA
AAGAATTAAAGATGGTTCCATTTCCTAGGCTACAGACAGGAATGGGGC
TCTAAATGGTTTTCATAGACTGGCTGTTAAAGGCCAAAAATTTTGGTAA
ATCAATGCTATATTATGCTCTTGAACTATTAAAACAGCCATAATTATTGT
CCCAAGATAGANNNNATATAGTCCTTTTTCAAAGATGATTATACGTGG
CTAGGTGACAGACATTAATGACTGACTCTGGAGAGTAAGTCATACCTG
CACTCTGTGGACTTGATGGTTCTTTTTCTAGAGCAAACAGAGCGTGGC
ATTTTGTTTTGACT
SEQ ID NO. 30:
TGTTTTCCCCTGTTAATGAGATATAGCTAGATATCGGTGTGTGTATTTC
TTTATTATTCTCTGGTTTTTGATCTGGCCTTGCCTCCAGGGCCAAACA
CTGATTTAGAAAGAGAGCCTTCTAGCTATTTTGGCATTGATGGCTTTTT
ATACCAGTGTGTCCAGTTAGATTTACTAGGCTTACTGACATGCTATTG
GTAAATCGCATTAAAGTTCATCTGAACCTTCTGTCTGTTGACTTCTTAG
TCCTCAGACATGGGCCTTTGTGTTTTAGAATATTTGAATTTGAGTTATT
GGGCCCCACTCCCTGTTTTTTATTAAAGAACGTGAGCCTGGGATACTT
TCA
SEQ ID NO. 31:
GACAGAAGATTAGCCACTCCTTGTGTAGGAAGTCAGGAACAGCTCCA
TTCCCCCAGCTCTCCCGGGCAGTATCAGAAGCCCCAGGTTGCCTGCT
GGGAGATGCATAATAAAGCTCAGTCCTGAACTAAACCAACACATCACC
TGGCCCTGGGTATAGAAGTAGTATTGTGAGGGGGATCTTGGGTCTTC
CAGGCCAGGTGTAAGCAAATGTAGGGAGTTCAGCCCCAGGAGAGATA
AAAGAATCATGCCATGGCCAGGTGCAGTGACTTATGCCTATAATCCCA
GCACTTTGGGAGGCCGAGATGGGTGGATCGCTTGAGCTTAGGAGTTC
GAGACCAGCCTGGGAAACATAGTGAAACCTCATCTCTACACACACAC
ACACACACACACACACACACACACACACACACACAAAGCCAGGTGTG
ATGGCATACATCTCTAGTCCC
SEQ ID NO. 32:
CAGAGGAGGCTAAGCCCGGGCAGCTACTTTGTTCCAGAAATCTAAGG
TCCCTGGAGNGAGGCTCTGCTTTNGGGAGGGGGAAGGGAGCTAACA
TTGCNGAGCACNAACTGTGAACCAGGTACAANTGGCAGAGCCTTTCC
ATACCTGTACTCACAACTAGCGGGTGAGGAGTCAAGGCAAATAGGTG
TCTCATAGCTCCCCATATCTCGGCAGTCGACCACCTCCTCCTTTGATT
CTCTGATGTCACTGCCAGTTCTCCTCCTATTGCTCTGACCTGTCTTTCT
CTGTGTCCTTTGCAAACTCATTCTCAACTCCTTAGACTCAGTCAAGTC
CCCCAGTTACACACTTCCATGGTACTATATATCATTCCTTCAGAGCACT
TAACACAGTTATTTCCTATGTATTTGTCCAGTCATTTGAATAATGATCC
TAGTTTCATTGGATGGAAAGTTCCACAAGGTCAGTGACCATTTCTATC
TGTGTTCACCAATGTGTTCCCAGTGCCCAGAAACAATGCCTAG

These probesets are AFFYMETRIX (HG-U133_PLUS_{—}2) probes (http://www.affymetrix.com/products_services/arrays/specific/hgu133plus.affx).

SEQ ID NO. 1 and 2 represents 2 isoformes of the ERBB2 genes. These 2 isoformes are matched by the probeset SEQ ID NO. 17.

SEQ ID NO. 5 and 6 represents 2 isoformes of the GRB7 gene. These 2 isoformes are matched by the probeset SEQ ID NO. 20.

SEQ ID NO. 8 and 9 represents 2 isoformes of the CRKRS gene. These 2 isoformes are matched by the probeset SEQ ID NO. 25.

SEQ ID NO. 10 and 11 represents 2 isoformes of the FGFR2 gene. These 2 isoformes are matched by the probeset SEQ ID NO. 27.

According to a particular embodiment of the invention, the method of the invention may be realized by hybridization of the polynucleotide sequences group comprising, or consisting of: SEQ ID NO. 17, SEQ ID NO. 18, SEQ ID NO. 19 and SEQ ID NO. 20.

According to another particular embodiment of the invention, the method of the invention may be realized by hybridization of the polynucleotide sequences group comprising, or consisting of: SEQ ID NO. 17, SEQ ID NO. 18, SEQ ID NO. 19 and SEQ ID NO. 20, and of SEQ ID NO. 31.

According to another particular embodiment of the invention, the method of the invention may be realized by hybridization of the polynucleotide sequences group comprising, or consisting of: SEQ ID NO. 17, SEQ ID NO. 18, SEQ ID NO. 19, SEQ ID NO. 20, SEQ ID NO. 21, and SEQ ID NO. 22.

According to another particular embodiment of the invention, the method of the invention may realized by hybridization of the polynucleotide sequences group comprising, or consisting of: SEQ ID NO. 17, SEQ ID NO. 18, SEQ ID NO. 19, SEQ ID NO. 20, SEQ ID NO. 21, SEQ ID NO. 22, and SEQ ID NO. 31.

According to another particular embodiment of the invention, the method of the invention may be realized by hybridization of the polynucleotide sequences group comprising, or consisting of: SEQ ID NO. 17, SEQ ID NO. 18, SEQ ID NO. 19, SEQ ID NO. 20, SEQ ID NO. 21, SEQ ID NO. 22, SEQ ID NO. 23 and SEQ ID NO. 24.

According to another particular embodiment of the invention, the method of the invention may be realized by hybridization of the polynucleotide sequences group comprising, or consisting of: SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24 and SEQ ID NO. 31.

According to another particular embodiment of the invention, the method of the invention may be realized by hybridization of the polynucleotide sequences group comprising, or consisting of: SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 28, SEQ ID NO. 30, SEQ ID NO. 31, SEQ ID NO. 32.

According to another particular embodiment of the invention, the method of the invention may be realized by hybridization of the polynucleotide sequences group comprising, or consisting of: SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32.

Advantageously, the method of the invention comprises the following steps:
a) reacting nucleic acids sample with a polynucleotide sequences group as described above, and
b) detecting the reaction product of step (a).

Advantageously, the nucleic acids sample may be labelled before reaction step (a).

Advantageously, the label of the polynucleotide sample may be selected from the group consisting of radioactive, colorimetric, enzymatic, e.g. biotinilated label, molecular amplification, bioluminescent or fluorescent labels.

Advantageously, the tissue may be fixed, paraffin-embedded, or fresh, or frozen.

For all the particular aspects of the invention, the expression of polynucleotide sequences in a tissue sample may by determined by measuring the expression level of RNA transcript(s) by real-time polymerase chain reaction (RT-PCR).

For all the particular aspects of the invention, the method may further comprise obtaining a control polynucleotide sample, reacting said control sample with said polynucleotide sequences, detecting a control sample reaction product and comparing the amount of said polynucleotide sample reaction product to the amount of said control sample reaction product.

Advantageously, the method the tissue sample may be a human sample.

Advantageously, the method of the invention allows to detect cancers selected from the group consisting of breast cancer, lung cancer, colorectal cancer, pancreatic cancer, prostate cancer, ovarian cancer, head and neck cancer, esophageal cancer, glioblastoma multiforme, hepatocellular cancer, gastric cancer, cervical cancer, liver cancer, bladder cancer, cancer of the urinary tract, thyroid cancer, renal cancer, carcinoma, melanoma, and brain cancer.

Advantageously, the tissue sample may be breast cancer sample.

Advantageously, the method of the invention allows the determination of the expression of the ERBB2 protein at cell membrane level.

Advantageously, the method of the invention allows to determine the ERBB2 immunohistochemical (IHC) status of a cancer patient, e.g., a breast cancer patient.

Another object of the invention is the use of the method of the invention for detecting, diagnosing, staging, monitoring cancer or following up the stage or aggressiveness of a cancer.

Any of the polynucleotide sequences groups as mentioned above may be used for the use according to the invention.

Advantageously, this use allows the monitoring of the treatment of a patient with a cancer selected from the group consisting of breast cancer, lung cancer, colorectal cancer, pancreatic cancer, prostate cancer, ovarian cancer, head and neck cancer, esophageal cancer, glioblastoma multiforme, hepatocellular cancer, gastric cancer, cervical cancer, liver cancer, bladder cancer, cancer of the urinary tract, thyroid cancer, renal cancer, carcinoma, melanoma, and brain cancer, e.g., breast cancer, and comprises the implementation of the method in any of its aspects on nucleic acids from a cancer tissue, e.g. breast cancer tissue sample of a patient.

Advantageously, the use of the method of the invention allows the assessment of the ERBB2 gene expression status of a patient for whose status could not has be previously clearly assessed with a immunohistochemical (IHC) assay for determination of ERBB2 overexpression in breast cancer, .e.g. of patients scoring 2+ with the HercepTest™ (Dako, Denmark, AS).

In other words, the use of this method allows the assessment of the ERBB2 gene expression status of a patient presenting equivocal results with IHC assay.

Indeed, a 2+ score obtained with the Herceptest™ does not allow to determine the ERBB2 status.

Advantageously, the monitoring relates to the clinical efficacy of an anti-ERBB2 treatment, e.g. by Herceptin™ (trastuzumab) treatment.

Advantageously, the use of the method allows the determination of a treatment for the patient or animal with a cancer according, e.g., breast cancer based on the analysis of differential gene expression profile obtained with said method.

Another object of the invention is a polynucleotide library useful for the molecular characterization of a cancer, e.g. breast cancer, that may comprise or may consist of polynucleotide sequences for detecting the genes as defined above.

Advantageously, the polynucleotide library may comprise, or may consist of cDNA total sequence or of cDNA subsequences of said genes.

Advantageously, the polynucleotide library may comprise, or may consist of primers allowing the detection of the genes mentioned above.

Advantageously, the polynucleotide library may comprise, or may consist of any of the groups of probesets as described above.

Advantageously, the polynucleotide library may comprise, or may consist, of: SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 28, SEQ ID NO. 30, SEQ ID NO. 31, SEQ ID NO. 32.

In any of these mode of realization, the polynucleotide library may be immobilized on a solid support.

In this case, the support may be selected from the group comprising nylon membrane, nitrocellulose membrane, glass slide, glass beads, membranes on glass support or silicon chip.

Another object of the invention is a kit comprising polynucleotide sequences, e.g., primers and probes, allowing the detection of the expression of the gene(s) and/or sequence(s) of the invention as defined above.

In a particular embodiment of the invention, the kit comprises a polynucleotide library as described above.

Any of the polynucleotide sequences groups as mentioned above may be used in the kit according to the invention.

The kit may comprise one or more of (1) nucleic acid extraction buffer/reagents and protocol; (2) reverse transcription buffer/reagents and protocol; and (3) qPCR buffer/reagents and protocol suitable for performing the method of the invention.

The kit may also comprise 1) data retrieval and/or analysis software.

The kit may be used by a laboratory or physician and be sent to a laboratory for sample testing, e.g., ISO-17025 MapQuant DX™ Lab Services at DNAVision SA (Gosselies, Belgium) on Affymetrix GeneChip® Systems 3000Dx2 (GCS3000Dx2), ensuring highly reproducible sample processing.

Another aspect of the invention relates to a report comprising a summary of the normalized expression levels of an RNA transcript or its expression products in a cancer cell obtained from a subject, wherein said RNA transcript is the RNA of a gene set select from one of the groups described above.

Another aspect of the invention relates to a report comprising a prediction of the response of a subject to treatment with an anti ERBB2 treatment, e.g. an ERBB2 antibody, based on the determination of the normalized expression levels of an RNA transcript or its expression products in a cancer cell obtained from the subject, wherein said RNA transcript is the RNA transcript of a gene group as described above.

Another object of the invention is a method for determining amplification of ERBB2 gene locus on chromosome 17q12-17q21.1 comprising determining the expression level of one or more RNA transcripts or their expression products in a biological sample containing cancer cells obtained from said subject, wherein the RNA transcript is of at least one, at two, at least three, or at least four, or at least five, or at least six, or at least seven, or of eight or larger group of genes selected from the group of genes located within less than one megabase on either side of ERBB2 gene on chromosome 17q12-17q21.1.

In said method, the gene(s) is (are) selected from ERBB2, C17orf37, GRB7, PERLD1, STARD3 and CRKRS. Advantageously, the method further include the hybridization of the tissue sample with the polynucleotide sequence SEQ ID NO. 31.

Another object of the invention is a method for predicting the response of a subject diagnosed with ERBB2 positive cancer to treatment with an ERBB2 inhibitor, comprising determining the expression level of one or more RNA transcripts or their expression products in a biological sample containing cancer cells obtained from said subject, wherein the RNA transcript is of one or more genes selected from the group consisting of ERBB2 and genes located near ERBB2 on chromosome 17g12-17q21.1, particularly the groups of genes as described above, notably the genes of table 1.

This method may further comprise the detection of the expression of SEQ ID NO. 31.

Unless otherwise noted, technical terms are used according to conventional usage.

In order to facilitate review of the various embodiment of the invention, the following explanation of specific terms is provided:

“Overexpression of polynucleotide sequences” means that the expression level of certain polynucleotide sequences is higher than the expression level of a control polynucleotide sequence.

“Underexpression of polynucleotide sequences” means that the expression level of certain polynucleotide sequences is lesser than the expression level of a control polynucleotide sequence.

There are many ways to collect quantitative or relative data on nucleic acids sequences, and the analytical methodology does not affect the utility of nucleic acids sequences expression in assessing the clinical outcome of a female mammal suffering from breast cancer. Methods for determining quantities of nucleic acids expression in a biological sample are well known from one of skill in the art. As an example of such methods, one can cite northern blot, cDNA array, oligo arrays, quantitative Reverse Transcription-PCR, e.g. real-time Real Time polymerase chain reaction (RT-PCR).

In the present invention, the term “polynucleotide” refers to a polymer of RNA or DNA that is single- or double-stranded, optionally containing synthetic, non-natural or altered nucleotide bases. A polynucleotide in the form of a polymer of DNA may be comprised of one or more segments of cDNA, genomic DNA or synthetic DNA.

Detection preferably involves calculating/quantifying a relative expression (transcription) level for each nucleic acids sequence.

By “ERBB2 amplicon”, in the sense of the present invention, is meant a wide region of amplification on chromosome 17q12-17q21.1, which contains many genes frequently amplified in breast tumours. This amplicon contains especially the ERBB2 gene.

By “genes”, in the sense of the present invention, is meant a polynucleotide sequence, e.g., isolated, such as deoxyribonucleic acid (DNA), and, where appropriate, ribonucleic acid (RNA). This sequence may be the complete sequence of the gene, or a subsequence of the gene that may be at least 90%, at least 95% identical to the complete gene sequence, which would be also suitable to perform the method of the analysis according to the invention. A person skilled in the art may choose the position and length of the gene by applying routine experiments. The term should also be understood to include, as equivalents, analogs of RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids. DNA may be obtained from said nucleic acids sample and RNA may be obtained by transcription of said DNA. In addition, mRNA may be isolated from said nucleic acids sample and cDNA may be obtained by reverse transcription of said mRNA.

By “polynucleotide sequences group consisting of”, in the sense of the present invention, is meant a group of polynucleotide sequences comprising exactly the polynucleotide sequences mentioned, and no polynucleotide sequence in addition nor in less than the polynucleotide sequences of the group.

By “cDNA total sequence of the gene”, in the sense of the invention, is meant the cDNA sequence resulting of the transcription of the DNA sequence coding for the gene.

By “cDNA subsequences of the gene”, in the sense of the invention, is meant a sequence of nucleic acids of cDNA total sequence of the gene that allows a specific hybridization under stringent conditions, as an example more than 10 nucleotides, preferably more than 15 nucleotides, and most preferably more than 25 nucleotides, as an example more than 50 nucleotides or more than 100 nucleotides.

The polynucleotide sample isolated from the subject and obtained at step (a) may be RNA, preferably mRNA. Said polynucleotide sample isolated from the patient can also correspond to cDNA obtained by reverse transcription of the mRNA, or a product of ligation after specific hybridization of specific probes to mRNA or cDNA.

The sequences SEQ ID No. 17 to SEQ ID NO. 32 are Affymetrix sequences (also refered hereafter as “probeset sequences”).

By “reacting nucleic acids sample with polynucleotide sequences”, in the sense of the invention, is meant contacting the nucleic acids sample with polynucleotide sequences in conditions allowing the hybridization of cDNA total sequence of the gene or of cDNA subsequences or of primers of the gene or of probeset sequences with polynucleotide sequences of the corresponding gene.

Animals corresponds to animals such as humans, mice, rats, guinea pigs, monkeys, cats, dogs, pigs, horses, or cows, preferably to humans, and most preferably to women.

Biological sample means any biological material, such as a cell, a tissue sample, or a biopsy from breast cancer.

A “Control” as used herein corresponds to one or more biological samples from a cell, a tissue sample or a biopsy from breast. Said control may be obtained from the same female mammal than the one to be tested or from another female mammal, preferably from the same specie, or from a population of females mammal, preferably from the same specie, that may be the same or different from the test female mammal or subject. Said control may correspond to a biological sample from a cell, a cell line, a tissue sample or a biopsy from breast.

DNA or RNA arrays consist of large numbers of respectively DNA or RNA molecules spotted in a systematic order on a solid support or substrate such as a nylon membrane, glass slide, glass beads or a silicon chip. Depending on the size of each DNA or RNA spot on the array, DNA or RNA arrays can be categorized as microarrays (each DNA or RNA spot has a diameter less than 250 microns) and macroarrays (spot diameter is grater than 300 microns). When the solid substrate used is small in size, arrays are also referred to as DNA or RNA chips. Depending on the spotting technique used, the number of spots on a glass microarray can range from hundreds to thousands.

Typically, a method of monitoring gene expression by DNA or RNA array involves the following steps:
a) obtaining a polynucleotide sample from a subject; and
b) reacting the sample polynucleotide obtained in step (a) with a probe immobilized on a solid support wherein said probe consist of polynucleotides having the nucleic acids sequence as previously described.
c) detecting the reaction product of step (b).

In the present invention, the term “immobilized on a support” means bound directly or indirectly thereto including attachment by covalent binding, hydrogen bonding, ionic interaction, hydrophobic interaction or otherwise.

Preferably, the polynucleotide sample obtained at step (a) is labeled before its reaction at step (b) with the probe immobilized on a solid support. Such labeling is well known from one of skill in the art and includes, but is not limited to, radioactive, colorimetric, enzymatic, e.g. biotinylation, molecular amplification, bioluminescent, electrochemical or fluorescent labeling.

Advantageously, the reaction product of step (c) is quantified by further comparison of said reaction product to a control sample.

Detection preferably involves calculating/quantifying a relative expression (transcription) level for each nucleic acids sequence.

Then, the determination of the relative expression level for each nucleic acid sequences previously described enables to assess the clinical outcome of the subject—i.e. female mammal—suffering from a cancer, e.g. a breast cancer, by the method of the invention.

The method of assessing the clinical outcome of a patient suffering from a cancer may further involve a step of taking a biological sample, preferably breast cancer tissue or cells from a patient. Such methods of sampling are well known of one of skill in the art, and as an example, one can cite surgery.

The provided method may also correspond to an in vitro method, which does not include such a step of sampling.

By “differential expression profile”, in the sense of the invention, is meant the difference between the level of expression of a gene in a control tissue, i.e. a breast tissue free of cancer, and the level of expression of the same gene in the sample analysed.

By “aggressiveness of a cancer”, in the sense of the invention, is meant, e.g., cancer growth rate or potential to metastasise. A so-called “aggressive cancer” will grow or metastasise rapidly or significantly affect overall health status and quality of life.

By “specificity”, in the sense of the invention, is meant the capacity, for a method, especially a diagnostic method, to exclude a disease (or a health problem), when it is really absent. The specificity is the proportion of healthy persons whose the result of the method or test is negative, calculated as follows: true negatives/(true negatives+false positives).

By “sensibility”, in the sense of the invention, is meant the capacity, for a method, especially a diagostic method, to detect a disease (or a health problem), when it really exists. The sensibility is the proportion of all the sick persons whose result to the method is positive, calculated as follows: true positives/(true positives+false negatives).

By “robustness”, in the sense of the invention, is meant the quality of being able to withstand changes in procedure or circumstances. It designs a method, or a group of genes, capable of coping well with variations (sometimes unpredictable variations) in its operating environment.

The method, and particularly the polynucleotide sequences groups of the invention, are “robust”, as it has been constructed by cross validations. It is furthermore independent of the subjective interpretation of a anatomo-pathologist.

For the classification of the patient in view of the ERBB2+ or ERBB2−, the man skilled in the art can use any method allowing the measurement of the expression of the genes of the invention. For example, the man skilled in the art can use the SVM method described in Vaknik et at. (Vapnik, 1998, Statistical Learning Theory. V. N. Vapnik. Wiley Interscience. The content of this document is hereby incorporated by reference.

The present invention will be understood more clearly on reading the description of the experimental studies performed in the context of the research carried out by the applicant, which should not be interpreted as being limiting in nature.

### BRIEF DESCRIPTION OF THE DRAWINGS

FIG. 1 “RESULT: HER2−” represents the probability of HER2 status depending on the HER2 score. The test returns the odds of having a HER2− or HER2+ tumor (y-axis) as a function of the predictive score (x-axis). The odds curves were calibrated using a reference set of 326 tumors with 15 (:)/0 of HER2+. Using an odds ratio threshold of 3:1 (outside of the grey zone), 95% of 2+ IHC tumors could be unambiguously classified.

FIG. 2 “QUALITY: OK” represents the deviation of the HER2 score. The Quality Control returns the maximal expected deviation of the HER2 predictive score (y-axis) as a function of the index quality (x-axis). The function was calibrated using 138 micorarrays hybridized with 42 different breast tumor samples submitted to various conditions. The index quality (p-value) tests the intra-chip reproducibility specifically for the 6 mRNAs that compose the HER2 predictive model.

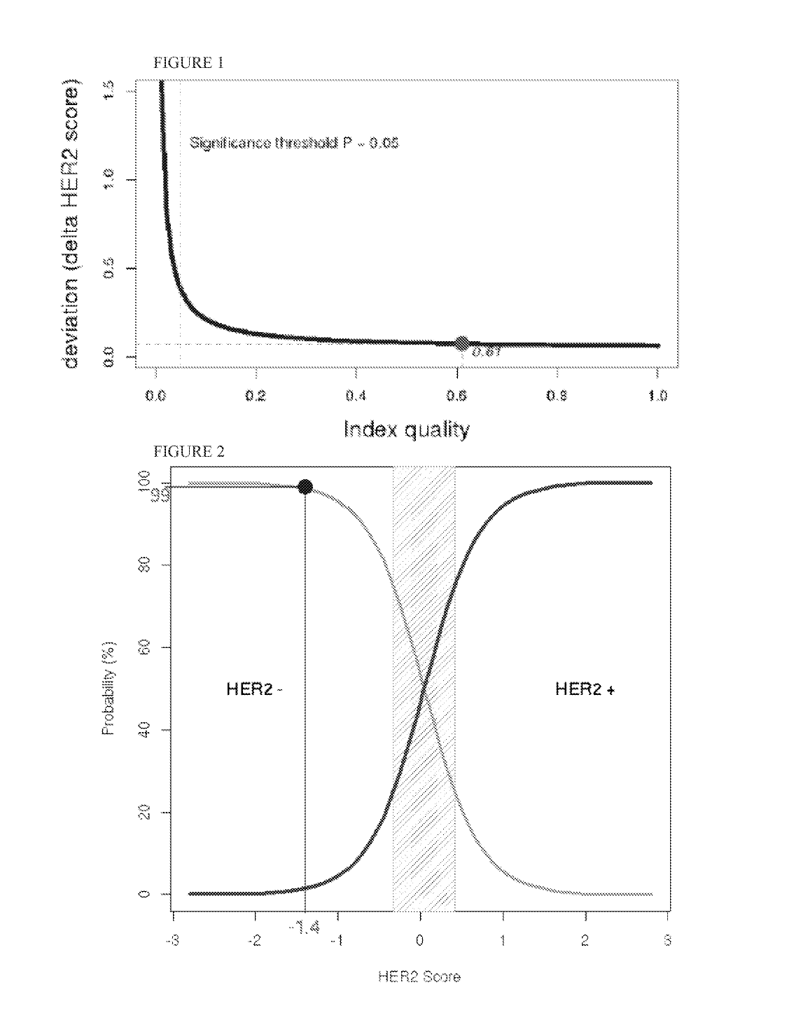

### EXAMPLES

### Example 1

### Material and Methods

The test has been developed on 152 tumor samples from Institut Paoli Calmettes (IPC) cancer Center: 126 IHC 0, 26 IHC 3+. These tumors have been profiled on an Affymetrix platform, HG-U133 plus 2.0 GeneChip®.

The HER2 signature has been obtained by the RFE-SVM (Recursive Feature Elimination-Support Vector Machine) classification method (Guyon et al. 2002; Machine Learning, 46, 389-422) by using the predefined set as the learning set.

We have used R Magpie implementation package (Ambroise, McLachlan). In order to guarantee robustness of our selection, we have used a cross validation protocol. We had first filtered absent probesets (expression level lower than 5.5 on the whole tumor set) and invariants (standard deviation lower than 0.5): those 2 probeset categories indeed tend to bring noise to classification.

### Results

The RFE-SVM algorithm provides an optimal signature with the 16 probesets: SEQ ID NO. 17, SEQ ID NO. 18, SEQ ID NO. 19 and SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32, of table 1.

The 16 probesets are located on the 17q12-17q21.1 locus except ZRANB1 and FGFR2 that both are on locus 10q26.

We have chosen the following 14 probesets among the 16 probesets: SEQ ID NO. 17, SEQ ID NO. 18, SEQ ID NO. 19 and SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 28, SEQ ID NO. 30, SEQ ID NO. 31, SEQ ID NO. 32, of table 1.

Performances have first been evaluated on 3 independent sets of tumors according to the following clinical criteria:

| Criteria | | IPC: | SET1 | | SET2 |
|---|---|---|---|---|---|
| Age | Mean | 54 | 51 | | — |
| | Range | 24-82 | 31-65 | | — |
| Grade | I | 7% | 20% | | 13% |
| | II | 17% | 41% | | 50% |
| | III | 52% | 28% | | 37% |
| | ND | 24% | 11% | | |
| Stade | 1 | 7% | 38% | | |
| | 2a | 12% | | | |
| | | | {close oversize brace} | 55% | |
| | 2b | 7% | | | |
| | 3a | 2% | | | |
| | | | {close oversize brace} | 3% | |
| | 3b | 1% | | | |

| | | | | |
|---|---|---|---|---|
| | 4a | | 1% | |
| | ND | 72% | 3% | |
| nodes | 0 | 12% | 0% | 77% |
| | 1-3 | 10% | 59% | 10% |
| | 4+ | 7% | 41% | |
| | ND | 72% | | 13% |
| Menopausal | YES | 13% | 41% | |
| status | NO | 15% | 58% | |
| | ND | ND | 1% | |
| ER | ER− | 40% | 17% | 17% |
| | ER+ | 36% | 74% | 80% |
| | ND | 24% | 9% | 3% |
| PR | PR− | 47% | 27% | 20% |
| | PR+ | 29% | 64% | 77% |
| | ND | 24% | 9% | 3% |
| HER2 | 0 | 52% | 76% | 53.5% |
| | 1+ | 5% | 0% | 27% |
| | 2+ | 1% | 3% | 13.5% |
| | 3+ | 11% | 12% | 3% |
| | ND | 31% | 9% | 3% |

Performances were already very satisfactory using our 16 probesets (Table 2):

TABLE 2

| | IPC: | | | | | |
|---|---|---|---|---|---|---|
| | Se | Sp | SET 1 | | SET 2 | |
| | (sensibility) | (specificity) | Se | Sp | Se | Sp |
| 16 probesets | 93% | 99% | 93% | 92% | 100% | 100% |

We have chosen to test the 14 probesets of the amplicon in order to understand the role of ZRANB1 and FGFR2. When doing that, we have globally improved the performance and validated the signature of the group of 14 probesets.

TABLE 3

| | IPC: | | SET 1 | | SET 2 | |
|---|---|---|---|---|---|---|
| | Se | Sp | Se | Sp | Se | Sp |
| 14 probesets | 93% | 99% | 93% | 94% | 100% | 100% |

This gene collection is particularly relevant since it covers ERBB2 amplicon from CRKRS to GRB7.

When comparing our 14 probesets signature to prior art signature or, to only one ARNm, we have noticed that we have improved it in terms of sensitivity, specificity and robustness.

TABLE 4

| | IPC: | | SET 1 | | SET 2 | |
|---|---|---|---|---|---|---|
| | Se | Sp | Se | Sp | Se | Sp |
| 14 probesets | 93% | 99% | 93% | 94% | 100% | 100% |
| Bertucci et al. (Oncogene. | 85% | 99% | 73% | 92% | 79% | 100% |
| 2004 Dec. 16; 23(58): | | | | | | |
| 9381-91) | | | | | | |
| ERBB2 | 93% | 93% | 93% | 90% | 100% | 99% |

### Conclusion

The method of the invention is an SVM model based on the expression of 14 probe sets corresponding to 6 genes of the 17q12 locus and one unknown sequence of the sequence of the 17q locus.

The test has been developed on 152 tumors and validated on 3 independent sets of 152 tumors. The test correlates with IHC method in 96% of cases and resolves equivocal cases (IHC 2+) in 95% of cases. We have also observed a concordance with FISH in more that 91% of cases but on a limited number of tumors (n=11).

### Example 2

### Material and Methods

We have validated our 14 probesets signature on 5 independent sets of tumors according to the following clinical criteria:

| Criteria | | IPC: | | SET 1 | SET 2 | SET 3 | | SET 4 |
|---|---|---|---|---|---|---|---|---|
| Age | Mean | 54 | | 51 | — | — | | — |
| | Range | 24-82 | | 31-65 | | — | | — |
| Grade | I | 7% | | 20% | 13% | 0% | | 20% |
| | II | 17% | | 41% | 50% | 21% | | 25% |
| | III | 52% | | 28% | 37% | 79% | | 55% |
| | ND | 24% | | 11% | | 0% | | |
| Stade | 1 | 7% | | 38% | | — | | — |
| | 2a | 12% | | | | — | | — |
| | | | {close oversize brace} | 55% | | | | |
| | 2b | 7% | | | | — | | — |
| | 3a | 2% | | | | — | | — |
| | | | {close oversize brace} | 3% | | | | |
| | 3b | 1% | | | | — | | — |
| | 4a | | | 1% | | — | | — |
| | ND | 72% | | 3% | | — | | |
| nodes | 0 | 12% | | 0% | 77% | 16% | | 49% |
| | 1-3 | 10% | | 59% | 10% | 42% | | 51% |
| | 4+ | 7% | | 41% | | 42% | | |
| | ND | 72% | | | 13% | | | — |
| Menopausal | YES | 13% | | 41% | | — | | — |
| status | NO | 15% | | 58% | | — | | |
| | ND | ND | | 1% | | — | | 42% |
| | ER− | 40% | | 17% | 17% | 26% | | 58% |
| ER | ER+ | 36% | | 74% | 80% | 74% | | |
| | ND | 24% | | 9% | 3% | | | — |
| | PR− | 47% | | 27% | 20% | 47% | | — |
| PR | PR+ | 29% | | 64% | 77% | 53% | | |
| | ND | 24% | | 9% | 3% | | | |
| | 0 | 52% | | 76% | 53.5% | 48% | | |
| | | | | | | | {close oversize brace} | 65% |
| HER2 | 1+ | 5% | | 0% | 27% | 5% | | |
| | 2+ | 1% | | 3% | 13.5% | 21% | | 6% |
| | 3+ | 11% | | 12% | 3% | 21% | | 27% |
| | ND | 31% | | 9% | 3% | 5% | | 2% |

From these 5 independent sets, 282 tumors have been selected based on their high-quality genomic profile, according to the criteria (average background, average noise, scale factor, percentage of present, gapdh, beta-actin and degradation slope of RNA) defined by Affymetrix (<<GeneChip® Expression Analysis Technical Manual>>, 2004) and which are generally applied in the art. As threshold we have chosen two standard deviation (which results in an alpha of 5% if the distribution is normal.) for each criterion.

For all these tumors, we have the detailed information IHC: 189 IHC 0, 22 IHC 1+, 20 IHC 2+, 51 IHC 3+.

Furthermore for IHC 2+, we have the FISH score expressed as positive or negative.

TABLE 6
HER2 IHC observed

| | | 0 | 1+ | 2+ | 3+ |
|---|---|---|---|---|---|
| HER2 Mapquant | Neg | 180 | 22 | 12 | 10 |
| predicted | Pos | 4 | 0 | 7 | 36 |
| | ND | 5 | 0 | 1 | 5 |

When comparing our 14 probesets signature to prior art signature or, to only one ARNm, regarding the 5 independent sets representing the 282 selected tumors, we have noticed that we had a good overall correlation but also in terms of sensitivity and specificity

TABLE 7

| | IPC: | | SET 1 | | SET 2 | | SET 3 | | SET 4 | |
|---|---|---|---|---|---|---|---|---|---|---|
| | Se | Sp | Se | Sp | Se | Sp | Se | Sp | Se | Sp |
| | (in %) | | | | | | | | | |
| 14 probesets | — | 100 | 92 | 95 | 100 | 100 | 100 | 100 | 69 | 100 |
| Bertucci et al. | — | 100 | 79 | 92 | 100 | 100 | 100 | 91 | 47 | 100 |
| (Oncogene. | | | | | | | | | | |
| 2004 Dec. 16; | | | | | | | | | | |
| 23(58): 9381-91) | | | | | | | | | | |
| ERBB2 | — | 93 | 93 | 91 | 100 | 99 | 100 | 100 | 78 | 91 |

### Conclusion

The test previously developed on 152 tumors, has been validated on 5 independent sets representing the 282 selected tumors. The test correlates with IHC method in 94% of cases with a global sensitivity and specificity of 78% and 98%, respectively. The test helps classify 271 tumors on 282 (96%). The test also helps resolve equivocal cases (IHC 2+) in 95% of cases (19/20). We also observe a concordance with FISH in 95% of cases (n=19).

Thus we have succeeded in 1-step test using our 14 probesets signature to globally improve the performance (sensitivity, specificity), compared to prior 2-steps tests such as those requiring performing the FISH score after performing IHC method.

1. A method for identifying ERBB2 alteration in tumors, in particular cancer, based on the analysis of the over or under expression of genes in a tissue sample, said analysis comprising :
the detection of the expression of a group of genes comprising, or consisting of: at least three, or at least four, or at least five, or at least six, or at least seven, or of eight genes of the ERBB2 amplicon, these genes being located within less than one megabase on either side of ERBB2, or
the detection of the expression of a group of genes comprising, or consisting of: at least three, or at least four, or at least five, or at least six, or at least seven, or of eight genes of the ERBB2 amplicon, these genes being located within less than one megabase on either side of ERBB2, and the gene corresponding to SEQ ID NO. 31.

2. A method according to claim 1, said group of gene comprising, or consisting of: at least three, or at least four, or at least five, or at least six, or at least seven, or of eight genes selected among the following genes: ERBB2, C17orf37, GRB7, PERLD1, STARD3, CRKRS, FGFR2, ZRANB1.

3. A method according to claim 2, said group of genes comprising, or consisting of: ERBB2, C17orf37, GRB7 and PERLD1.

4. A method according to claim 2, said group of genes comprising, or consisting of: ERBB2, C17orf37, GRB7, PERLD1 and STARD3.

5. A method according to claim 2, said group of genes comprising, or consisting of: ERBB2, C17orf37, GRB7, PERLD1, STARD3 and CRKRS.

6. A method according to claim 2, said group of genes comprising, or consisting of: ERBB2, C17orf37, GRB7, PERLD1, STARD3 , CRKRS and the gene corresponding to SEQ ID NO. 31.

7. A method according to claim 1, said detection being realized by hybridization of polynucleotide sequences from a tissue sample with cDNA total sequence or with cDNA subsequences of said genes, or with the following polynucleotide sequences: SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32.

8. A method according to claim 1, said detection being realized by hybridization of polynucleotide sequences from a tissue sample with a polynucleotide sequences group comprising or consisting to: SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 28, SEQ ID NO. 30, SEQ ID NO. 31, SEQ ID NO. 32.

9. A method according to claim 1 comprising:
a) reacting nucleic acids sample with polynucleotide sequence according to claim 7 or 8, and
b) detecting the reaction product of step (a).

10. The method according to claim 9, wherein said nucleic acids sample is labelled before reaction step (a).

11. The method according to claim 10, wherein the label of the polynucleotide sample is selected from the group consisting of radioactive, colorimetric, enzymatic, molecular amplification, bioluminescent or fluorescent labels.

12. The method according to claim 10, wherein the label is an enzymatic label, e.g., a biotinilated label.

13. The method according to claim 1, wherein said tissue is fixed, paraffin-embedded, or fresh, or frozen.

14. The method according to claim 1, wherein the expression is determined by measuring the expression level of RNA transcript(s) by real-time polymerase chain reaction (RT-PCR).

15. The method according to claim 9, further comprising obtaining a control polynucleotide sample, reacting said control sample with said polynucleotide sequences, detecting a control sample reaction product and comparing the amount of said polynucleotide sample reaction product to the amount of said control sample reaction product.

16. The method according to claim 1, wherein said tissue sample is a human sample.

17. The method according to claim 1, wherein said cancer is selected from the group consisting of breast cancer, lung cancer, colorectal cancer, pancreatic cancer, prostate cancer, ovarian cancer, head and neck cancer, esophageal cancer, glioblastoma multiforme, hepatocellular cancer, gastric cancer, cervical cancer, liver cancer, bladder cancer, cancer of the urinary tract, thyroid cancer, renal cancer, carcinoma, melanoma, and brain cancer.

18. The method of claim 17, wherein tissue sample is breast cancer sample.

19. A method according to claim 1, for determining the expression of the ERBB2 protein at cell membrane level.

20. A method according to claim 1, for determining the ERBB2 immunohistochemical (IHC) status of a cancer patient, e.g., a breast cancer patient.

21. Detecting, diagnosing, staging, monitoring cancer or following up the stage or aggressiveness of a cancer, using the method of claim 1.

22. Monitoring the treatment of a patient with a cancer selected from the group consisting of breast cancer, lung cancer, colorectal cancer, pancreatic cancer, prostate cancer, ovarian cancer, head and neck cancer, esophageal cancer, glioblastoma multiforme, hepatocellular cancer, gastric cancer, cervical cancer, liver cancer, bladder cancer, cancer of the urinary tract, thyroid cancer, renal cancer, carcinoma, melanoma, and brain cancer, e.g., breast cancer, comprising the implementation of the method of claim 1 on nucleic acids from a cancer tissue, e.g. breast cancer tissue sample of a patient.

23. Assessing the ERBB2 gene expression status of a patient for whose status has been previously assessed with a immunohistochemical (IHC) assay for determination of ERBB2 overexpression in breast cancer, .e.g. of patients scoring 2+ with the HercepTest™ (Dako, Denmark, AS) using the method of claim 21.

24. Monitoring according to claim 21, wherein said monitoring relates to the clinical efficacy of an anti-ERBB2 treatment, e.g. by Herceptin™ (trastuzumab) treatment.

25. Determining a treatment for the patient or animal with a cancer according, e.g., breast cancer based on the analysis of differential gene expression profile obtained with said method of claim 1.

26. A polynucleotide library useful for the molecular characterization of a cancer, including breast cancer consisting of polynucleotide sequences group for detecting the genes defined in claim 1.

27. A polynucleotide library according to claim 26, consisting of cDNA total sequence or of cDNA subsequences of said genes.

28. A polynucleotide library according to claim 26, consisting of the following sequences: SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 28, SEQ ID NO. 30, SEQ ID NO. 31, SEQ ID NO. 32.

29. A polynucleotide library according to claim 26, consisting of the following sequences: SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32.

30. A polynucleotide library according to claim 26, immobilized on a solid support.

31. A polynucleotide library according to claim 26, wherein the support is selected from the group comprising nylon membrane, nitrocellulose membrane, glass slide, glass beads, membranes on glass support or silicon chip.

32. A kit comprising a polynucleotide library according to claim 26.

33. A method for determining amplification of ERBB2 gene locus on chromosome 17q12-17q21.1 comprising determining the expression level of one or more RNA transcripts or their expression products in a biological sample containing cancer cells obtained from said subject, wherein the RNA transcript is of at least one, at two, at least three, or at least four, or at least five, or at least six, or at least seven, or of eight or larger group of genes selected from the group of genes located within less than one megabase on either side of ERBB2 gene on chromosome 17q12-17q21.1.

34. A method according to claim 33 wherein for the group of genes comprising, or consisting of: ERBB2, C17orf37, GRB7, PERLD1, STARD3 , CRKRS and the gene corresponding to SEQ ID NO. 31.

35. A method for predicting the response of a subject diagnosed with ERBB2 positive cancer to treatment with an ERBB2 inhibitor, comprising determining the expression level of one or more RNA transcripts or their expression products in a biological sample containing cancer cells obtained from said subject, wherein the RNA transcript is of one or more genes selected from the group consisting of ERBB2 and genes located near ERBB2 on chromosome 17q12-17q21.1

36. The method of claim 35 wherein the one or more genes are groups of one, two, three, four, five, six, seven or eight genes selected among the genes of table 1.

37. The method of claim 36 further comprising the detection of the expression of SEQ ID NO. 31.
